(19) United States
(12) Patent Application Publication
(10) Pub. No.: US 2019/0192825 A1
(43) Pub. Date: Jun. 27, 2019

(54) PASSIVE RELEASE SAFETY SHIELD FOR CATHETER ASSEMBLIES AND RELATED METHODS

(71) Applicant: B. Braun Melsungen AG, Melsungen (DE)

(72) Inventor: Kit Weng Neoh, Penang (MY)

(21) Appl. No.: 16/211,093

(22) Filed: Dec. 5, 2018

Related U.S. Application Data
(60) Provisional application No. 62/610,661, filed on Dec. 27, 2017.

(51) Int. Cl. A61M 25/06; A61M 25/00

(52) U.S. Cl. CPC A61M 25/0618; A61M 25/02; A61M 25/0606; A61M 25/0014

(57) ABSTRACT

Safety shields and safety shield housings are described for intravenous (IV) catheter assemblies for preventing needlestick. A safety shield can cover a tip of a needle upon separation from a catheter hub. A safety shield housing can be provided between a catheter hub and a needle hub of the catheter assembly, and the safety shield housing can house the safety shield. The catheter hub can be a straight catheter hub or can include a side fluid port for use in as an integrated IVC.

### FIELD OF ART

The current device, system and method relate to safety shields, and specifically to passive safety shields for intravenous (“IV”) catheters. In particular, the device, system, and method relate to a safety shield, to cover a needle tip, having a biasing spring, such as a spring mechanism, and a housing for the safety shield.

### BACKGROUND

Catheters are commonly used for a variety of infusion therapies. For example, catheters are used for infusing fluids, such as normal saline solution, various medicaments, and total parenteral nutrition, into a patient, withdrawing blood from a patient, or monitoring various parameters of the patient's vascular system.

Following placement of the catheter into the vasculature of a patient, an IV fluid source can be connected to the catheter adapter or catheter hub, opening the blood control valve. Thus connected, fluid from the IV source can begin flow into a patient through the catheter.

In order to place the catheter into the vasculature of a patient, a needle is used and then separated from the catheter. To prevent accidental needle stick, safety shields or needle guards are used to cover the tip of the needle after separation from the catheter.

### SUMMARY

The present disclosure provides for a catheter assembly having a catheter hub having a catheter; a safety shield or needle guard; a safety shield housing; and a needle hub having a needle; wherein the safety shield housing is disposed between the catheter hub and the needle hub; wherein the safety shield is configured to couple with a proximal portion of the catheter hub. The safety shield can be biased by the needle to then couple to the proximal portion of the catheter hub. The safety shield located inside the safety shield housing can engage the catheter hub in the ready to use position.

In exemplary embodiments, a catheter assembly having a catheter hub having a catheter; a safety shield or needle guard; a safety shield housing; and a needle hub having a needle; wherein the safety shield housing is disposed between the catheter hub and the needle hub; wherein the safety shield comprises a hook housed inside the safety shield housing; wherein the hook latches onto a proximal portion of the catheter hub.

Methods of making catheter assemblies and of using the catheter assemblies as described herein are within the scope of the present invention.

Aspects of the present disclosure can include a catheter assembly comprising a catheter hub comprising a catheter; a needle guard; a safety shield housing having an interior having the needle guard located therein; and a needle hub comprising a needle with a needle tip; wherein the safety shield housing is disposed between and couples the catheter hub and the needle hub and the needle guard engaging the catheter hub.

The safety shield housing can receive a portion of the catheter hub at a distal opening of the safety shield housing.

The needle guard can comprise a hook and the hook can engage a proximal portion of the catheter hub.

The hook can have at least one of a curved surface or a lip for engaging the catheter hub.

The needle guard can comprise a resilient spring.

The needle guard can comprise a proximal hole and a distal hole spaced from one another by an intermediary wall, the proximal hole and the distal hole being sized to accept the needle.

The needle guard can comprise a resilient spring that is biased in a position where the needle is inserted through the proximal hole and the distal hole; and wherein the resilient spring of the needle guard can be unbiased in a position where the needle is displaced out of the distal hole.

The longitudinal axis of the distal hole can be offset from a longitudinal axis of a proximal hole when the needle guard is unbiased.

The needle guard can have a first part and a second part and wherein the first part of the needle guard can be located in a slot between a ramp and a bottom side of the safety shield housing.

The second part of the needle guard can be located in a slot between the ramp and a proximal side of the safety shield housing.

The needle guard can comprise a proximal hole, a distal hole, a hook, and a folded biasing portion comprising at least two acute angles or arcuate surfaces.

The catheter hub can comprise a side fluid port and a tubing connected to the side fluid port.

A valve or a septum can be located inside the catheter hub.

A fluid adapter can be connected to the tubing, which can connect to a side fluid port.

Aspects of the present disclosure comprise a method of assembling a catheter assembly. The method of assembling can comprise coupling a safety shield housing with a catheter hub comprising a catheter tube; inserting a needle guard comprising a resilient spring into an interior of the safety shield housing; and coupling a needle hub comprising a needle with the safety shield housing; wherein the safety shield housing is disposed between and couples the catheter hub and the needle hub and the needle guard engaging the catheter hub.

The method can include receiving a portion of the catheter hub through a distal opening of the safety shield housing.

Various aspects of valved catheter assemblies and components thereof are discussed in PCT patent applications PCT/EP2016/069619 and PCT/EP2016/069643, the contents of which are expressly incorporated herein by reference.

An IV catheter assembly may more broadly be referred to as a needle assembly or a needle device can comprise a catheter hub with a catheter tube and a bushing. The bushing can be configured, such as sized and shaped, to wedge the proximal end of the catheter tube against the interior wall surfaces of the catheter hub to retain the catheter tube to the catheter hub.

Interiorly of the catheter hub, a septum or valve, an actuator or valve opener, if a valve is used and not a septum, and a needle guard, such as a safety shield or tip protector, are provided. The needle guard can be located proximally of the valve or septum.

A needle, which can include a change in profile proximal of a needle tip, can be inserted through the proximal opening of the catheter hub with the needle tip protruding from the distal opening of the catheter tube in a ready to use position. The catheter hub can be made from a single hub body as shown or from two or more hub bodies assembled together. The catheter hub can alternatively be an integrated catheter hub with a tubing line and a fluid adapter. The catheter hub can still alternatively be a catheter hub with a side port, such as a ported catheter, for flushing using a male Luer connector.

A cannula hub or needle hub can attach to the proximal end of the needle and can contact the proximal end of the catheter hub when assembled thereto in the ready to use position. The proximal opening of the catheter hub can be sized with a female Luer taper, optionally with external threads, to engage with a male Luer tip in a Luer slip or a Luer lock.

The needle guard, which is understood to include structural features for guarding the needle tip from unintended needle sticks, is configured to be removed with the needle following successful venipuncture. The valve and valve actuator, the latter is incorporated, are configured to remain with the catheter hub for controlling fluid flow therethrough. The actuator is configured to be pushed distally by a male tip into the valve to open the valve for fluid flow. Further information regarding an exemplary needle guard is discussed in U.S. Pat. No. 8,568,372, the contents of which are expressly incorporated herein by reference.

In alternative embodiments, the needle guard or shield can embody multiple components that cooperate to block the needle tip from unintended needle sticks. For example, the needle guard can comprise a spring loaded needle carrier located inside a safety barrel, said needle carrier having a needle attached thereto. Following successful venipuncture, a release tab can be pressed to release the spring to then move the needle carrier and needle inside the protective barrel to block the needle tip from unintended needle sticks.

A flash back plug or blood stopper assembly can be connected to the needle hub to stop blood flow out the flashback chamber of the needle hub. The flash back plug can be provided at the proximal end the needle hub to allow air to vent but stops blood from spilling out the proximal end of the body of the flash back plug, which can have a chamber and a hydrophobic filter assembled to the chamber. Alternatively, a syringe can be attached to the proximal end of the needle hub. A second valve and actuator can also be placed within the needle hub.

A protective cap with a sleeve and a saddle can be provided to cover the needle during packaging and before use, which is conventional. The saddle can surround at least part of the catheter hub and the needle hub and be removably engaged to the needle hub. The cap should be removed from the needle assembly before use. The catheter hub can be provided with a pair of wings to facilitate securement of the catheter hub to a patient following use.

A catheter assembly provided in accordance with other aspects of the present invention includes an integrated intravenous catheter assembly or integrated IVC. The catheter assembly comprises a catheter hub and a catheter tube, a needle hub having a needle extending through the catheter tube with a needle tip extending distally of the catheter tube opening. A crimp or change in profile formed with the needle can be located proximally of the needle tip and inside the catheter tube in the ready to use position.

The catheter hub has a side fluid port, and a fluid adaptor attached to the fluid port by a tubing having a lumen for fluid flow between the side fluid port and the adaptor. As shown, the fluid adaptor is a Y-site comprising at least one needleless female Luer connector. The other opening of the Y-site can have a conventional vent plug. Optionally, the fluid adaptor can be a female needleless adapter.

The proximal end of the catheter hub can be equipped with a septum, a seal or a valve and prevents flow thereacross after removal of the needle and the needle hub following successful venipuncture.

A needle shield or guard can be incorporated between the needle hub and the catheter hub of the integrated IVC. The needle guard can be placed inside a safety shield housing and the combination needle guard and safety shield housing can be placed between the needle hub and the catheter hub, as further discussed below. The catheter hub can include a pair of wings. The needle hub can alternatively have a wing that extends distally along a side of the catheter hub opposite the side of the fluid port.

Following successful venipuncture, the needle and needle hub can be removed from the catheter hub and the catheter tube and the needle tip can be covered or protected by the needle guard, and optionally with the safety shield housing or retracted inside a protective barrel. Blood flow can be stopped or restricted by a seal or septum located at the proximal end of the catheter hub, proximal of where the side fluid port opens into the catheter hub so as to not interfere with fluid flow through the side fluid port. IV fluid or other medicaments can flow into the catheter hub via connection to the fluid adapter.

An IV catheter assembly provided herein can have a first state with the safety shield in place in a ready to use position in which the needle tip extends out the distal opening of the catheter tube and a second state where the needle hub is removed from the catheter hub following use. After removal of the needle hub, a male Luer tip, such as a syringe tip, can be inserted into the proximal opening of the catheter hub to push open the valve with the valve opener or a male Luer tip can be connected to a fluid adapter of an integrated IVC.

An IV catheter assembly with a valve inside a catheter hub, which in an embodiment can have a two-part hub body. Further information regarding the catheter assembly having the valve is discussed in U.S. Pat. No. 9,114,231, the contents of which are expressly incorporated herein by reference. In other examples, a ported catheter assembly or an integrated catheter assembly having an integrated tubing can be practiced with the valve and valve opener described herein.

A distal hub element or first hub part of the catheter hub can have a holding section, such as a nose section, in which a catheter tube can be held, such as with a metal bushing. The proximal end of the first hub element or part can have an enlarged diameter relative to the nose section of the distal end portion and forms a connecting section for connecting with a rear hub element or second hub part.

The two-part hub configuration can facilitate assembly of a valve and a valve opener, as further discussed below. However, catheter assemblies described herein are not limited to a two-part hub as a singularly formed hub or three or more hub parts may be used to form a catheter hub without deviating from the scope of the catheter assemblies described herein.

The distal end of the rear hub part can overlap the proximal end of the front or first hub part and which is provided at its proximal end with a female Luer fitting with exterior threads, forming a female threaded Luer. In some examples, the threads can be omitted and the proximal opening can function as a female Luer slip. Between the two hub parts, a valve in the form of a check valve shaped as a cylinder with a valve disk, valve disc, or a valve disk without a skirt is inserted and is fixed in place by the two hub elements.

In other embodiments, a single hub body is used and the valve is held in place by placing the valve next to or against a shoulder in the interior of the hub element. A second shoulder may be incorporated to secure the valve in place with adhesive or bonding being optional to further assist holding the valve in place. In still yet other examples, a single catheter hub body is used with internal shoulders or undercuts incorporated for retaining the valve therein, without adhesive or bonding. In still another embodiment of the present disclosure, the valve is held in the catheter hub by an interference fit.

In the ready position of the catheter assembly, a nose section of a needle hub can be inserted into the catheter hub. A hollow needle can be fixed to the nose section of the needle hub and extends through the valve, the catheter hub, and the catheter tube so that the needle tip can be exposed beyond the tapered end of the catheter tube.

Between the needle hub and the valve and inside the catheter hub, a valve opening device, valve actuator, or valve opener with a nose end for opening the valve can be slidably or displaceably arranged. In an example, the nose end of the valve opener can have a truncated cone-shaped locating section or a tapered section. In other examples, the valve actuator can have a nose section with a radiused tip, a square tip, or a tip with angular surfaces.

On the proximal side of the valve actuator, a plunger section or pusher end can be provided. The plunger section is sized and shaped to be pushed by the male Luer conical fitting of a medical implement, such as a syringe tip or an IV tubing adaptor, to open the valve. The pusher end can adjoin the nose end. The pusher end can have a surface to be pushed. The pusher end can have one or more gaps for fluid flow thereacross. In an example, the nose section can incorporate a groove to facilitate engagement with the valve so that even if the medical implement no longer pushes on the valve actuator, the nose section remains engaged with the valve to keep the valve in an open position for fluid flow.

As explained above, a typical exchange of elements could be the use of the valve opener. In one example, there is only a single element or leg forming the pusher end. In another example, two legs with a hollow space therebetween or a gap are provided. The two proximal ends of the two legs can provide a surface to be pushed by the medical implement. The space between the two legs can be sized to receive a safety shield, or needle guard. For example, the safety shield can be positioned in the space between the two legs. In still other examples, the pusher end can be a cylinder having one or more openings through the cylinder to provide space for engagement with a needle guard, which can engage the catheter hub through the one or more openings or engage an edge of each of the openings of the cylinder, if more than one opening.

In other examples, a third housing having a cavity can be positioned between the catheter hub and the needle hub such as the several examples described herein including one involving an integrated IVC. The safety shield or needle guard may be positioned in the cavity of the third housing and the third housing having mechanical features to engage the catheter hub and the needle hub. For example, the third housing can have an extension that contacts the interior or the exterior of the catheter hub to secure the third housing to the catheter hub and/or to prevent early activation of the needle guard. In some examples, the needle guard is omitted and the catheter assemblies only incorporate a valve and a valve opener. In still other examples, the valve opener can be omitted and the valve is sized and shaped to open or close based on fluid pressure.

On withdrawal of the hollow needle from the catheter hub following successful venipuncture, a change in profile provided near the needle tip and having the form of a radial projection on the hollow needle, such as by crimping, can engage with a perimeter defining the proximal opening on the rear wall or proximal wall of the safety shield so that the safety shield can be removed from the catheter hub with the needle.

As the needle tip moves proximal of the two distal walls of the two arms, the two arms move, such as spring or deflect radially to disengage from the interior of the catheter hub. As the arms of the safety shield move radially, the arms, or the distal walls of the arms, can cover the needle tip to prevent unintended needle sticks. In other examples, the change in profile can include a sleeve, a notch, or a material buildup on the shaft of the needle.

Subsequent to removal of the needle following successful venipuncture, the two or more flaps of the valve, due to their elastic properties, close the one or more slits through the depth of the valve disk or disc so that no blood or substantially no blood can flow out through the catheter. Valves provided herein can include three slits starting from the middle of the valve and extending radially over a short radial distance towards the outer perimeter to form elastic flaps that can be expanded by the hollow needle and closed when the needle is removed. In some examples, the flaps can remain open and engaged with a valve opener, which can be referred to as a one-time use valve. The number of slits defining the number of flaps can vary, ranging from one slit to more than four slits.

A syringe tip of a syringe can abut the pusher end of the valve actuating element and pushes it against the valve so that the nose section of the valve opener advances against the flaps of the valve to open the slits thereby opening the valve so that liquid can flow there-across.

As shown, the nose end of the actuator can be inclined. Thus, as the syringe is removed and the forward force on the actuator is removed, the elasticity of the material of the valve can be sufficient for the two or more flaps to uncoil and push the actuator in the proximal direction to close the seal. The valve therefore can automatically close upon withdraw of the pushing force on the actuator and can be re-used by inserting a male Luer tip into the catheter hub to again advance the valve opener into the valve. In other examples, the valve and valve opener can be of a single use type such that once the valve opener is advanced by the syringe tip to open the valve, the valve opener remain engaged with the valve, even after removal of the syringe tip.

The catheter hub can include a shoulder that acts as a proximal stop for the actuator when the flange on the actuator abuts the shoulder, which defines the proximal most position of the flange of the actuator. In other examples, the second hub section may incorporate other structural features, such as a tapered internal cavity, to stop the proximal travel of the actuator. A second shoulder just proximal of the first shoulder can be incorporated. If so, the two shoulders can define a groove. The two radially outer areas of the spring arms of the needle guard, which may be referred to as elbows between the distal walls and the two elongated sections of the two arms, can be configured to abut the second shoulder in the ready position.

When the needle hub with the hollow needle is removed from the catheter hub, the safety shield can be held generally stationary by the shoulder until the change in profile, such as a crimp, near the needle tip comes to abut on the rear proximal wall of the needle guard and the needle tip moves proximally of the two distal walls on the safety shield. At this point, the two spring arms, which are no longer restrained in the radial direction by the needle, spring inwards to cover the needle tip and separate from the second shoulder, whereupon the safety shield with the hollow needle can be removed from the catheter hub. Further information regarding the safety shield is discussed in U.S. Pat. No. 7,736,339, the contents of which are expressly incorporated herein by reference.

A catheter assembly provided herein can include a safety shield housing or needle guard housing located between the catheter hub and the needle hub. Further, while the catheter hubs shown in the various figures illustrate a standard straight catheter hub, the catheter hub can be an integrated catheter hub as shown in FIG. 1A, which has a side fluid port, a tubing, and a fluid adapter.

The catheter assembly generally comprises a catheter tube or, its shorthand version, a catheter and a catheter hub at a distal end of the catheter assembly. A distal end of the safety shield housing, which can comprise a housing structure and a cover, can contact a proximal end of the catheter hub. The safety shield housing can house the needle guard. For example, the needle guard can be spaced from the catheter hub when using the safety shield housing. This is in contrast with the embodiment wherein the needle guard is housed inside the catheter hub. In this way, the catheter hub does not have the design constraints of having to be sized and shaped to receive and house a safety shield. The needle hub or cannula hub can attach to a proximal end of the needle and can contact a proximal end of the safety shield housing when assembled thereto in the ready to use position.

The change in profile of the needle can be configured to engage with an outer circumference defining a proximal opening on the rear wall or proximal wall of the needle guard so that the needle guard, and hence the safety shield housing, can be removed from the catheter hub with the needle. The proximal wall of the needle guard can be slidably located between the location of the change in profile and the proximal end of the needle. Thereby, the proximal wall of the needle guard can be prevented from passing the change in profile and from becoming separated from the needle. Further explanation of the interaction between the needle and the needle guard is discussed below with respect to FIGS. 9-12.

The catheter hub can be a wingless catheter hub, a winged catheter hub, a ported catheter hub, an integrated catheter assembly or a catheter adapter with an inner lumen with a valve or permanent septum.

A needle hub can have an orientation guide. The orientation guide can embody a tab and extend from a distal end of the needle hub in a distal direction to provide a surface for the safety shield housing to align therewith. The orientation guide can be a substantially rectangular feature for contacting a side of the safety shield housing. In some examples, the safety shield housing can have a recess or channel to receive the orientation guide, which can seat flushed within the recess or project above the recess.

The orientation guide can ensure proper orientation of the needle relative to the safety shield housing. This can ensure that the safety shield housing is oriented upward such that a distal wall can be properly oriented. Additionally, this can ensure access to a hope on the safety shield housing. Further, the safety shield housing can then orientate relative to the catheter hub, thus indirectly the needle, especially the needle bevel at the needle tip, can orientate relative to the catheter hub. The bevel preferably faces up, away from the patient, when in the ready to use position.

The assembled catheter assembly can allow for the catheter hub to be partially retained inside an interior of the safety shield housing in ready to use position.

The cover of the housing can be inset to be flush with the edges of the sides of the safety shield housing. The cover can snap fit into the side opening of the housing. Optionally, the cover can be glued to the safety shield housing.

A safety shield housing provided herein can have two parts or can comprise two parts, which can include a housing structure and a cover. The housing structure can have a number of distinct sides. In an example, the distinct sides can include a proximal side, a distal side, a top side, a lateral side, and a bottom side. The top side, the lateral side, and the bottom side can define a channel or recess that can constitute an interior of the safety shield housing. With the proximal side and the channel or recess, a cavity is defined for accommodating a needle guard. The open side of the channel or recess, or the side opposite the lateral side, can be configured to be coupled with the cover or to receive the cover.

The exterior surfaces of the proximal side, the top side, the lateral side, and the bottom side can each be substantially flat. Alternatively, one or more of the surfaces can have curvature or another geometric shape. In embodiments, the housing structure can have a cylindrical exterior. Optionally, surface features can be provided on the exterior of the housing structure, such as bumps or projections for gripping purposes.

The distal side can extend from the top side. In an example, the distal side can extend outwardly from the housing structure. The distal side can have a fillet bending radially of the surface of the top side. The distal side can act as a stop or a finger tab for a user's finger to push against for leverage when using the needle device. The distal side can have an outer edge or free end that points away from the needle when the housing structure is mounted onto the needle.

The top side can further comprise a hole located at an intermediary location of the top side. The hole can be used to position a needle guard, as described elsewhere herein. The hole can be sized to accommodate a desired safety shield or needle guard setting tool to provide access to the interior of the safety shield housing for manipulating the needle guard with the setting tool when installing the needle guard in the safety shield housing.

The lateral side can have a notch portion at a distal end. The notch portion can be viewed as a cut-out on the wall surface of the lateral side. The notch portion can have two edges having a generally L-shape, which can form the cut-out on the wall surface of the lateral side.

Part of the lateral side without the cut-out can extend approximately to the distal side extending from the top side. However, the part of the lateral side having the notch portion can be shorter and can define an axially shorter portion or wall surface of the lateral side. In an example, the bottom side can extend evenly with the axially shorter portion of the lateral side.

In the interior of the housing structure, there can be a proximal region and a distal region. The proximal region can have stop pieces. The stop pieces may embody plates or bands forming thicker portions of the bottom side and top side, respectively, that can act as an insertion limiter for the catheter hub when the catheter hub is inserted from a distal end of the safety shield housing in an assembled or ready position.

The stop pieces can be generally rectangular materials or pieces extending inwardly from the top side and the bottom side but short of the distal ends or distal edges of the stop side and the bottom side. Thus, the top side and the bottom side each comprises a section of greater thickness at the proximal region, due to the stop pieces, than at the distal region. The stop pieces can be unitarily formed with the top side and the bottom side, such as by plastic injection molding.

Surfaces of the stop pieces may be generally parallel to the top side and the bottom side. The stop pieces can be inset by a distance from at least one edge of the top side and the bottom side of the housing structure. In an example, both the top side and the bottom side have an inset so that the housing structure is said to have two insets.

The insets can be provided along the open side of the housing structure for receiving the cover. The inset can be provided to aid in configuration for coupling of the cover, as described below, so that the cover seats flushed with the edges of the housing structure.

The distal region can have a catheter ramp on the bottom side. The catheter ramp can extend from a distal end of the housing structure to one of the stop pieces. The catheter ramp can be configured to aid in guiding the catheter hub in axial movement. The top side can also have a catheter ramp. In the illustrated embodiment, part or all of the catheter ramp can have a surface with an arcuate section or shape that can correspond to an arcuate guide for a cylindrical catheter hub. The catheter ramp can be shaped to correspond to an exterior shape of the catheter hub.

The interior of the housing structure can be configured to accommodate a needle guard.

The housing structure can be configured to fit with the cover or to receive the cover such that the cover covers the open side of the housing structure to cover the interior. The cover can fit on a side of the housing opposite of the lateral side of the housing structure so that in the assembled state, the edges of the cover seat within the insets in a flushed configuration.

The cover can have a cover side and a fitment tab. The fitment tab can include a catheter ramp, which can have a surface with an arcuate section to provide an arcuate guide for a cylindrical catheter hub. The fitment tab can extend a length that is shorter than the width of the cover. For example, the fitment tab can extend from a distal portion of the cover while leaving the proximal portion of the cover on the side of the fitment tab free or open of any tab. The catheter ramp can be shaped to correspond to an exterior shape of the catheter hub. The fitment tab can be configured to fit into the open distal region of the bottom side distal of the stop piece and the thickness of the cover, such as the edges of the cover, can fit into the inset adjacent to the stop pieces.

The cover side of the cover can fit between the top side and the bottom side of the housing structure and forming one of the sides thereof. Alternatively, the cover side can abut the side edges of the top side and the bottom side. The cover can have a length that matches the bottom side, and not fit within the insets. Alternatively, the cover can be shorter or longer than the housing structure.

A needle guard provided herein can have a proximal wall, an intermediary wall, a distal wall, and a hook. The needle guard can be unitarily formed from a stamped metal piece and then manipulated or cold worked to a final shape. In an example, the metal is stainless steel and the intermediary wall and/or distal wall can act as resilient arms, spring arms, or resilient springs, such as cantilever springs. In other examples, the needle guard can be made from two or more pieces that are assembled or welded together. The multiple pieces can be made from plastic and metal materials.

An end of the proximal wall can join with an end of the intermediary wall. Another end of the intermediary wall can join with an end of the distal wall. Another end of the distal wall can join with a hook. The proximal wall can be substantially perpendicular to the intermediary wall with some angular variation being optional. The proximal wall can be configured to be substantially perpendicular to a longitudinal axis of the needle when the needle guard is mounted to the safety shield housing and the combination mounted onto the needle.

At intermediary locations of the proximal wall and the distal wall, there can be perimeters defining a proximal hole and a distal hole, respectively. The proximal hole and the distal hole can have diameters that are slightly larger than an outer diameter of the needle configured to pass through the proximal hole and the distal hole. In an example, the proximal and distal holes can have a half to three thousandths total clearance with the needle diameter with a different range contemplated. In other examples, the distal hole can be larger than the proximal hole and the distal hole can allow a needle crimp to pass thereacross, as further discussed below. The holes can be circular or round or can have a shape that is other than round, such as polygonal.

The proximal hole and the distal hole can be arranged such that in a relaxed state, such as an unbiased state of the needle guard, a longitudinal axis of the proximal hole is offset from a longitudinal axis of the distal hole. For example, elevation-wise from the surface of the intermediary wall, the center of the distal hole can be higher than the center of the proximal hole. The proximal hole and the distal hole can be arranged such that when the needle guard is in a biased, or compressed state, the proximal hole and the distal hole can be aligned.

In an example, the centers of the proximal and distal holes are about at the same height, elevation-wise, from the intermediary wall when the needle guard is in the biased state. Viewed differently, the longitudinal axis of the proximal hole can align with the longitudinal axis of the distal hole when the needle guard is in the biased state.

In an example, the proximal hole can be sized and shaped to have a dimension or a diameter sufficiently small to catch a stop or change in profile, such as a crimp or a weld bead, on the needle during withdrawal of the needle following venipuncture. This relative sizes of the change in profile and the proximal hole opening can create an interference and can prevent the needle guard from separating distally off of the needle when the needle hub is withdrawn from the catheter hub. Alternatively, the proximal hole can have an extended emboss configured to catch the change in profile, or crimp, on the needle.

The distal wall of the needle guard can be sized and shaped to flex between a biased position and an unbiased position. The distal wall can have the ability to flex such that the end of the distal wall joined with the hook can be deflected with movement along a plane substantially perpendicular to the longitudinal axis of the needle.

The distal wall can be formed with a curve shaped portion, whereby the curve shape allows for elastic buckling spring effect to occur with the distal wall. The curve shaped portion can join with the intermediary wall with an acute angle. The distal hole can be located on the curve shaped portion. The curve shaped portion can join at another end with a flat portion. The flat portion can be substantially perpendicular to the longitudinal axis of the needle.

The distal wall can include both the curve shaped portion and the flat portion. The flat portion can join with the hook. In an example, the curve shaped portion has a larger surface area than the area of the flat portion. In other examples, the relative surface area sizes of the two portions can be reversed.

The hook can be composed of a first portion and a hooking portion. The hooking portion can be a lip or a tab for engaging the catheter hub. The hook can alternatively have a curved surface or an arcuate surface for engaging the catheter hub. Optionally, the hook can have at least one of a lip and a curved surface. The hook can be configured to allow for hooking or latching on to a Luer thread on a catheter hub or a corresponding detent formed on the catheter hub. Such a detent formed on the catheter hub can take the form of a flange, Luer locking ears, a notch, a slot or a peg.

The first portion can be configured to extend in a direction towards a distal end of the catheter hub in order for the hooking portion to clear the corresponding Luer thread or detent of the catheter hub for hooking.

The hook can have different geometries as desired and necessary for interfacing with a corresponding catheter hub. In embodiments, the hook can be triangular in shape with an upward angled first portion and a downward angled hooking portion. In embodiments, the hook can have a hooking portion without a first portion. For example, the hook can be comprised of an arcuate portion or a curved portion.

The needle guard or safety shield can be made of thin metal sheet being stamped into the desired shape and then cold work or formed, such as bent, into final shape wherein, in the biased position, the safety shield engages the catheter hub and in the unbiased position the safety shield disengages the catheter hub.

Although the needle guard can be assembled with the safety shield housing, it is also understood that the needle guard can be assembled with a catheter assembly without a safety shield housing. For example, the needle guard can be applied in a suitably sized and shaped catheter hub with a Luer taper or needle hub without the safety shield housing. For example, such a catheter hub can have an interior catch for coupling with the needle guard.

In the un-activated position, the hook can couple to a latching portion of the catheter hub, thereby preventing premature activation of the needle guard. The engagement between the hook and the latching portion also allows the catheter hub and the safety shield housing to remain together until separation of the needle guard from the catheter hub.

The first or upper ramp can be spaced from the stop piece at the top side by a slot or gap. The first ramp can also be spaced from the proximal side of the housing structure by a rear slot or gap. For example, the upper ramp can attach to the lateral side and can extend from the lateral side towards the open side of the housing structure and can be spaced from the stop piece and the proximal side by gaps.

Similarly, the second or bottom ramp can attach to the lateral side and can extend from the lateral side towards the open side of the housing structure. The second ramp can be spaced from the stop piece at the bottom side by a slot or gap and spaced from the proximal side by a rear slot or gap. As, further discussed below, the gap between the second ramp and the bottom side can be configured to receive the intermediate wall of the needle guard when mounting the needle guard to the housing structure. The proximal wall of the needle guard can be configured to fit into the rear gaps adjacent the proximal side.

The ramps of the housing can act as guides for the needle by maintaining the needle therebetween and also provide a slot or gap for the placement of the needle guard. The intermediary wall of the needle guard can be positioned between the bottom ramp and one of the stop pieces. The proximal wall of the needle guard can be positioned between the top and bottom ramps and the proximal side of the safety shield housing, at rear gaps. The proximal wall and the intermediary wall of the needle guard can be firmly fitted in the slots between the ramps, proximal side, and the stop piece.

The needle guard can be said to comprise a first part and a second part and wherein the first part of the needle guard is located in a slot between a ramp and a bottom side of the safety shield housing and the second part of the needle guard is located in a slot between the ramp and the proximal side of the safety shield housing. In an example, the dimension of the rear gap between the second ramp and the proximal side is sized to fit the proximal wall of the needle guard to hold the needle guard securely in place. In an example, the dimension of the gap between the second ramp and the stop piece is also sized to fit the proximal wall of the needle guard to hold the needle guard securely in place. The proximal wall can be held in a tight fit between the second ramp and the proximal side and between the second ramp and the stop piece.

The size, shape, and location of first, upper, or top ramp of the housing can also prevent the sharp distal tip of the needle from pivoting a sufficient amount to be able to be tilted upwards towards the distal hole on the distal wall of the needle guard in an unbiased position of the needle guard to prevent re-exposing the needle tip through the distal hole.

The first and second ramps can extend distally forward in the safety shield housing such that the ramps contact the distal wall of the safety shield in a biased position. The ramps can act as a hard stop for compression or biasing of the needle guard. In other examples, the ramps can extend short of or not touch the distal wall 804 in the safety shield biased position.

Alternatively, the ramps can be formed and/or molded on the cover instead of the housing structure. Said differently, in an alternative embodiment, the ramps can separate from the housing structure. The ramps can be placed into the interior of the housing structure when mounting the cover to the open side of the housing structure.

In an example, the ramps can be integrally molded as a single component together with the housing structure of the safety shield housing. The ramps can be molded in a separate step from molding either the housing structure or the cover. The two ramps can be similar in size and shape or can be different.

The proximal wall and the intermediary wall of the needle guard can be substantially perpendicular to one another. In this way, the needle guard can be retained in the safety shield housing. In other examples, the proximal wall and the intermediary wall of the needle guard are located less than or greater than 90 degrees from one another to thereby create a biasing effect against the structures of the safety shield housing when mounted within the gaps. The intermediary wall can be retained in the slot or gap between the bottom ramp and one of the stop pieces. The material thickness of the needle guard can fit into the slots or gaps in a size-on-size fit or with a slight clearance that enables some movement or shifting of the needle guard relative to the housing structure.

The distal wall of the needle guard can be positioned in the distal region of the safety shield housing, such that the distal wall can move between a biased state and an unbiased state. The top ramp and the bottom ramp can have distal ends that have tapered distal end surfaces configured to approximately match the contour of the curved shape portion of the needle guard in the biased state of the needle guard. Optionally, the distal end surfaces of the top and bottom ramps are not tapered.

In an un-activated, biased position, the curve shaped portion of the needle guard can be constrained by the needle 108. That is, when the needle shaft passes through the proximal hole on the proximal wall and the distal hole on the distal wall, the needle can constrain the distal wall against the intermediary wall are create a biasing or resilient force between the distal wall and the needle. In this way, the curve shaped portion can be flexed, or biased, such that the distance from the hook to the intermediary wall can be shorter than when the needle guard is unbiased by the needle.

As shown, a hole for accessing the interior of the safety shield housing with a tool can be positioned above the hook to facilitate assembly of the needle through the distal hole.

When the access hole is so positioned, a tool can be inserted into the hole at the top side of the housing structure to bias the needle guard, such as to align the proximal hole and the distal hole. A needle can then pass through the distal hole and retain the needle guard in the biased state or position without the tool. In the biased position, the second portion or hooking portion of the hook contacts the catheter hub and retains the catheter hub from separation in combination with the catheter ramp. This can make it difficult for the intended user to remove the catheter hub from the safety shield housing.

The needle can be guided by a proximal hole of the proximal side of the safety shield housing, the proximal hole of the needle guard, and optionally the top ramp. In the un-activated, biased position, the needle can be further guided by the distal hole and the catheter hub and/or the catheter tube.

In embodiments, there may be additional ramps sized and shaped in the interior of the safety shield housing to hold or support the catheter hub to prevent unwanted longitudinal or transverse movement of the catheter hub from the safety shield housing. The additional ramps can also be incorporated to support the needle during retraction of the needle.

The additional ramps can also be incorporated to ensure encapsulation of the needle within the safety shield housing by further limiting the needle's ability to pivot or rotate. The additional ramps can be molded within the cover or the housing structure or can be added, similar to adding one or more shims or tabs.

A gap can be provided between the hook and the interior surface of the top side. The gap can be provided as a clearance gap to enable the distal wall to spring outwardly when no longer biased by the needle, such as after retraction of the needle tip proximally of the distal hole. The gap should have a clearance or dimension larger than the thickness of the second portion of the hook, or other structure of the hook if embodied in a different shape, to enable the second portion to travel radially to clear the latching portion. Once the hook separates from the latching portion, catheter hub can separate from the needle guard and the safety shield housing.

When the needle hub and the needle are retracted from the proximal end of the catheter hub, such that the needle is retracted from the distal hole, the needle guard can be released from the biased position. The biased curve shaped portion can return to an unbiased position, or to a less biased position, wherein the distance from the hook to the intermediary wall is longer than when in the biased position.

When the sharp distal tip of needle is retracted, or moved proximally, into the needle guard during the withdrawal of the needle, the needle no longer extends through the distal hole on the distal wall of needle guard. The distal wall of the needle guard can flex to its unbiased, activated position.

During this unbiased movement of the needle guard, the hook at the top of the distal wall of needle guard releases itself upwards from the catheter hub to clear the latching portion, allowing the catheter hub to be disconnected from the needle guard.

As the distal hole of the distal wall of the needle guard is no longer aligned with the longitudinal axis of the needle, the distal wall of the needle guard can act as a barrier to the sharp distal tip of the needle. The needle can be contained within the needle guard where the proximal hole of the safety shield catches the crimp of the needle while the distal wall of the needle guard acts as a barrier against distal movement of the needle tip to re-expose the needle tip. In this protected position, the needle tip is trapped inside an enclosed space defined by the safety shield housing and the distal wall.

The crimp can prevent the retracted needle from retracting from the proximal hole, or the needle guard and safety shield housing from displacing distally off of the needle tip. As the needle no longer constrains the safety shield in a biased position through the distal hole, the longitudinal axis of the distal hole is offset from the longitudinal axis of the proximal hole and from the longitudinal axis of the needle. This prevents the needle from sliding forward past the distal wall of the needle guard after retracting from the distal wall.

With the needle guard in the unbiased position, the catheter hub is uncoupled, and is slidably movable relative to the needle guard and the safety shield housing. This allows for separation of the catheter hub from the safety shield housing. Accordingly, the catheter hub can now be use for infusion or for drawing samples. In some examples, the catheter hub is provided with a valve and a valve opener so that fluid flow can be stopped by the valve until the slits of the valve are opened by the valve opener.

The latch can connect the cover side and the top side. The latch can be located at an intermediary location along the length of the top side. Alternatively, the latch can be near the distal end or the proximal end of the top side.

The latch can be defined by a thinner portion of material relative to the adjacent areas of the cover and the housing structure. The thinner portion of material of the latch allows for a relatively weaker area of material that allows for flexure. As such, the latch can deform when force is applied to the cover and the housing structure to allow for rotation of one portion relative to the other.

In embodiments, the latch can be a material having different characteristics than at least one of the cover and the housing structure. For example, the latch can be a polymer with a lower modulus of elasticity than the material of at least one of the cover or the housing structure.

In embodiments, the cover, the housing structure, and the latch can each be of different materials from one another.

In still other examples, the latch can be an elastomer glued or welded to the cover and the top side. In an example, the latch can have the same material as the cover and the housing but is made from a thinner material or can include one or more notches or weakened sections or strips to facilitate bending.

According to methods of forming the safety shield housing, a single step injection molding can be performed where the different materials are co-molded or insert molded to form the safety shield housing with different materials among the cover, the housing structure, and the latch.

Embodiments can be formed by integrally molding of the cover, the housing structure, and the latch. Alternatively, embodiments can have the cover and the housing structure formed separately and then joined by the latch. The latch can be joined to the cover and the housing structure in such embodiments by means such as gluing, bonding, or welding.

Also, the latch can be a separate component. The cover and the housing structure can each have a through-hole configured to receive ends of the latch. The ends of the latch can have retention components such that each end can be coupled to and retained in the through-holes of the cover and the housing structure.

An embodiment of a safety shield housing in which the cover and the housing structure are coupled by a latch that is a living hinge is also disclosed. The cover, housing structure, and living hinge can be integrally formed by molding. The living hinge can be defined by a thinner portion of material relative to the adjacent areas of the cover and housing structure. The thinner portion of material of the living hinge allows for a relatively weaker area of material that allows for flexure. As such, the living hinge can deform when force is applied to the cover and the housing structure to allow for rotation of one portion relative to the other

The living hinge can extend along an entire length of an edge of the top side. Alternatively, the living hinge can extend only along a portion of the edge. In embodiments where the living hinge extends only along a portion of the edge, the living hinge can be substantially located at a proximal portion of the edge, a distal portion of the edge, or an intermediary portion of the edge. The living hinge can extend less than half of the length of the edge, half, or extend more than half of the length of the edge.

In embodiments, the living hinge can couple the cover and the housing structure at an edge of the bottom wall instead. Alternatively, in embodiments where the cover is a different wall of the safety shield housing, the living hinge can be attached to one of the edges of the adjacent walls of the housing structure to which the cover contacts.

Alternatively, embodiments can have the cover and the housing structure substantially formed first separately and then joined by molding of the living hinge and surrounding areas of the cover and housing structure.

In embodiments, the living hinge can be a material having different characteristics than at least one of the cover and the housing structure. For example, the living hinge can be a polymer with a lower modulus of elasticity than the material of at least one of the cover or the housing structure. In embodiments, the cover, the housing structure, and the living hinge can each be of different materials from one another.

A safety shield housing where the top wall, the bottom wall, and the proximal wall are split in two housing sections is also disclosed. In an example, the safety shield housing is split along the lengthwise direction into two pieces. The two housing sections can be coupled by a latch coupling a proximal side of one housing section to a proximal side of another housing section. The safety shield housing can be split along a central axial plane such that the two housing sections are substantially equal in size. Alternatively, the safety shield housing can be split along a line offset from the central axial plane. In some examples, one of the housing sections, which may be called a first housing section and a second housing section, can be viewed as a cover and the other housing section can be viewed as a housing structure.

Each half of the housing structure can include a top side, a proximal side, a distal side, and a bottom side. On interior surfaces of the top side and the bottom side, there can be provided stop pieces. The stop pieces can be inset from edges of the top side and the bottom side by a mating region. The stop pieces, the top ramp, and the bottom ramp of the second housing section can protrude beyond the central axial plane of the housing structure. As such, the protruding portions of the stop pieces, the top ramp, and the bottom ramp can fit into the mating region when the covering and the housing structure are fitted together in an assembled state.

The hole can be split into arcuate portions of the second housing structure of the first housing structure. In embodiments, the hole can be offset from the divide between the two housing sections so that the hole is located completely on one of the housing sections.

The latch can be composed on a material suitable for the movement of bringing the two housing sections into an assembled position. The latch can have an arcuate portion. The latch can be comparatively thinner than the thickness of the sides of the two housing sections. In embodiments, the latch can be attached to the two housing sections at a position near the central axial plane.

In embodiments, the latch can be a material having different characteristics than at least one of the two housing sections. For example, the latch can be a polymer with a lower modulus of elasticity than the material of at least one of the two housing sections. In embodiments, the two housing sections and the latch can each be of different materials from one another.

Embodiments can be formed by integral molding of the latch with the two housing sections. The method can include conventional molding techniques, can include co-molding, can include insert molding.

Alternatively, embodiments can have the housing sections formed separately and then joined by the latch. The latch can be joined to the two housing sections in such embodiments by means such as molding or plastic welding. Also, the latch can be a separate component. The two housing sections can each have a through-hole in the respective proximal sides configured to receive ends of the latch. The ends of the latch can have retention components such that each end can be coupled to and retained in the through-holes of the two housing sections.

The various alternative safety shield housings can be used to house a needle guard described elsewhere herein, such as the needle guard described elsewhere herein. Features in the alternative safety shield housings for accommodating a needle guard, such as incorporating slots or gaps, can be similar to those described elsewhere herein.

A needle guard with a folded biasing portion connected to the distal wall can be practiced. The needle guard can be similar to the needle guard described elsewhere with a few exceptions. In an example, the folded biasing portion can be formed from a plurality of folds between the distal hole and the intermediary wall.

The proximal wall can have a proximal hole and the distal wall can have a distal hole. The plurality of folds create a spring portion. The folds can have sharp angled folds or can be undulating like a sine wave. The folded biasing portion can include a first acute angle between the intermediary wall and a first fold portion, a second acute angle, a second fold portion, and an obtuse angle between the distal wall and the second fold portion. In other examples, the folded biasing portion can include additional fold portions and additional acute angles.

The first acute angle and the second acute angle can be biased into smaller angle biased positions and can allow for compression of the distal wall to shorten the distance between the distal hole and the intermediary wall.

The distal wall can have a shape and size such that it is able to flex between a biased position and an unbiased position. The distal wall can have the ability to flex such that the end of the distal wall joined with the hook can be deflected with movement along a plane substantially perpendicular to the longitudinal axis of the needle.

The proximal hole and the distal hole can be arranged such that in a relaxed, unbiased state of the needle guard, a longitudinal axis of the proximal hole is offset from a longitudinal axis of the distal hole. The proximal hole and the distal hole can be arranged such that when the needle guard is in a biased, or compressed state, the proximal hole and the distal hole can be aligned such that the longitudinal axis of the proximal hole aligns with the longitudinal axis of the distal hole.

The needle guard can be assembled with a safety shield housing, but can also be assembled to a catheter assembly without a safety shield housing. The needle guard can be applied in a suitably sized and shaped catheter hub or needle hub without the safety shield housing. For example, such a catheter hub can have an interior catch for coupling with the needle guard.

An assembled catheter assembly can have a bottom ramp sized to provide clearance for the folded biasing portion of the needle guard. As such, the bottom ramp can be smaller than the top ramp.

A hook of a needle guard can couple to the latching portion of the catheter hub, thereby preventing separation of the catheter hub from the safety shield housing.

The ramps of the housing can act as guides for the needle and also provide a slot for the placement of the needle guard. The intermediary wall of the needle guard can be positioned between the bottom ramp and one of the stop pieces. The proximal wall of the needle guard can be positioned between the top and bottom ramps and the proximal side of the safety shield housing \. The proximal wall and the intermediary wall of the needle guard can be firmly fitted between the ramps, proximal side, and the stop piece.

The top ramp of the housing can also prevent the sharp distal tip of the needle from being able to be tilted upwards towards the distal hole on the distal wall of the safety shield in an unbiased position to re-expose the needle following needle protection.

Alternatively, the ramps can be formed and molded on the cover instead of the housing structure side of the safety shield housing. The two ramps can be have different shapes to accommodate the folded biasing portion of the needle guard. The lower ramp can extend distally not as far as the upper ramp.

The ramps can be integrally molded as a single component together with the housing structure of the safety shield housing. The ramps can be molded in a separate step from the molding of either the housing structure or the cover.

The proximal wall and the intermediary wall can be substantially perpendicular to one another or can be other than right angle to create a bias against the safety shield housing when the needle guard is installed inside safety shield housing. In this way, the needle guard can be retained in the safety shield housing in the rear gap between the ramps and the housing proximal wall. A portion of the length of the intermediary wall can be retained by the bottom ramp and one of the stop pieces, such as the stop piece on the bottom wall. Alternatively, the proximal wall and the intermediary wall can meet with an oblique angle. The bottom ramp can be correspondingly sized and shaped to maintain retention of the needle guard.

The distal wall of the needle guard can be positioned in the distal region of the safety shield housing, such that the distal wall can be biased and unbiased.

In an un-activated, biased position, the folded biasing portion of the needle guard can be biased by the needle. In this way, the folded biasing portion is flexed, or biased, such that the distance from the hook to the intermediary wall is shorter than when the needle guard is unbiased, or not constrained by the needle.

The access hole can be positioned above the hook. A tool can be inserted into the hole of the top side to bias the needle guard such that the needle can pass through the distal hole in addition to the proximal hole to bias the needle guard. In the biased position, the hooking portion can contact the latching portion of the catheter hub and retains the catheter hub from separation from the needle guard and the needle shield housing. This can make it difficult for the user to remove the catheter hub from the safety shield housing prior to activating or releasing the needle guard from the biased state.

The needle can be guided by a proximal hole of the proximal side of the housing, the proximal hole of the needle guard, and the top ramp. In the un-activated, biased position, the needle can be further guided by the distal hole and the catheter hub.

In embodiments, there may be additional ramps sized and shaped in the interior of the safety shield housing to hold or support the catheter hub to prevent unwanted longitudinal or transverse movement of the catheter hub from the safety shield housing. The additional ramps may be similar to shims used for adjusting the various components to take up slacks or gaps.

When the needle hub and the needle are retracted from the proximal end of the catheter hub, such that the needle is retracted from the distal hole, the needle guard can be released from the biased position. The folded biasing portion can return to an unbiased position, or less biased position, wherein the distance from the hook to the intermediary wall is longer than the biased position.

During this unbiased movement, the hook at the top of the distal wall of needle guard can move upwardly of the latching portion of the catheter hub, allowing the catheter hub to be disconnected from the needle guard.

As the distal hole of the distal wall of the needle guard is no longer aligned with the longitudinal axis of the needle, the distal wall of the needle guard can act as a barrier to the sharp distal tip of the needle. The needle can be contained within the safety shield where the proximal hole of the safety shield catches the crimp of the needle while the distal wall of the needle guard provides a barrier for the tip of the needle.

The crimp can prevent the retracted needle from moving proximally of the proximal hole or the needle guard to displace distally off of the needle tip. As the needle is no longer retaining the safety shield in a biased position through the distal hole, the longitudinal axis of the distal hole can be offset from the longitudinal axis of the proximal hole and the longitudinal axis of the needle. This prevents the needle from sliding forward past the distal wall of the needle guard after retracting from the distal wall.

With the needle guard in the unbiased position, the catheter hub is uncoupled, and can slidably move relative to the needle guard and the safety shield housing. This allows for separation of the catheter hub from the safety shield housing. Accordingly, the catheter hub can now be ready for infusion. The catheter hub can include a valve and a valve opener.

A needle guard described herein can have a design derived from the safety shield spring clip disclosed in E.P. Patent No. 1,702,643, which is expressly incorporated herein by reference in its entirety.

The needle guard can have two resilient arms defined by intermediary walls. The two intermediary walls of the two arms can be respectively joined at their proximal ends in a hinged arrangement to the ends of a proximal wall.

The proximal wall can be configured to be substantially perpendicular to a longitudinal axis of the needle. The proximal wall can have a proximal hole having a diameter that is slightly larger than an outer diameter of the needle configured to pass through the proximal hole but smaller than a crimp or change in profile incorporated with the needle.

The intermediary walls of the two arms can each include a proximal wide portion, an intermediary narrow portion, and a distal wide portion. The two arms can act as cantilever springs. The intermediary walls or the two arms can be intermediary narrow portions on opposed lateral sides, such that the intermediary walls of the two arms can cross one another.

One of the intermediary walls can attach to the distal wall. The distal wall can have a cut out portion with a lateral clamping edge. The distal wall can be attached to a hook. The hook can be composed of a first portion and a hooking portion. The hook can be configured to allow for hooking or latching on to a Luer thread on a catheter hub or a corresponding latching portion formed on the catheter hub. Such a detent formed on the catheter hub can take the form of a flange, Luer locking ears, a notch, a slot or a peg.

The first portion can be configured to extend in a direction towards a distal end of the catheter hub in order for the hooking portion to clear the corresponding Luer thread or detent of the catheter hub.

The hook can have different geometries as desired and necessary for interfacing with a corresponding catheter hub. In embodiments, the hook can be triangular in shape with an upward angled first portion and a downward angled hooking portion. In embodiments, the hook can have a hooking portion without a first portion. For example, the hook can be comprised of an arcuate portion or a curved portion.

The other intermediary wall can have an angled distal end section. The distal end of the angled distal end section can have a lateral clamping edge. The angled distal end section can be angled in a direction towards the cutout portion.

The safety shield can be made of a stamped metal sheet that is then folded into the desired designed shape wherein, in the biased position, the safety shield engages the catheter hub and in the unbiased position the safety shield disengages the catheter hub.

The needle guard can be applied in a suitably sized and shaped catheter hub or needle hub without the safety shield housing. For example, such a catheter hub can have an interior catch for coupling with the needle guard.

A catheter assembly can generally comprise a catheter or catheter tube and a catheter hub at a distal end of the catheter assembly. A distal end of the safety shield housing with a housing structure and a cover can contact a proximal end of the catheter hub. The safety shield housing can house the needle guard. This is in contrast with other embodiments wherein the needle guard is housed in the catheter hub. In this way, the catheter hub does not have the design constraints of having to be sized and shaped to receive and house a safety shield. The needle hub or cannula hub can attach to a proximal end of the needle and can contact a proximal end of the needle guard when assembled thereto in the ready to use position.

As described above, embodiments of the components from the incorporated references can be implemented for the various components disclosed herein. For example, regarding the catheter hub, it is possible to practice a wingless catheter hub, a winged catheter hub, a ported catheter hub, or a catheter adapter with an inner lumen with a valve or permanent septum.

A needle can bias the intermediary walls such that the needle can extend into the catheter hub. In this case, lateral clamping edges on each of the distal wall and the angled distal end section can rest on the needle in the biased position.

The needle guard can be held in place by fitment or containment in the safety shield housing with the top ramp, bottom ramp, and interior walls of the safety shield housing retaining the proximal wall of the needle guard, as previously discussed. The top ramp can be sized and shaped, such as a trapezoid, to fit with the needle guard. The bottom ramp can be sized and shaped, such as a rectangle, to fit with the needle guard.

In the un-activated, biased position, the needle can retain and bias the intermediary wall resilient arms. The shaft of the needle passes through the proximal hole and the lateral clamping edges on each of the distal wall and the angled distal end section can rest on the needle in the biased position. In this position, where the distal wall is biased relatively downward, the hook, with a first portion and a hooking portion, can latch onto a latching portion of the catheter hub.

The angled distal end section of a needle guard can contact the distal wall and block a direct sight path from the proximal hole to the cut out portion.

When the sharp distal tip of needle is retracted, or moved, into the needle guard during the withdrawal of the needle, the needle no longer extends through the cut out portion on the distal wall of needle guard. The distal wall of needle guard can flex to its unbiased, activated position.

During this unbiased movement, the hook at the top of the distal wall of needle guard can release itself upwards from the catheter hub, allowing the catheter hub to be disconnected from the needle guard.

As the cut out portion of the distal wall of the needle guard has a lowest opening that prevents clearance of the needle, the distal wall of the needle guard can act as a barrier to the sharp distal tip of the needle. The needle can be contained within the safety shield where the proximal hole of the safety shield catches the crimp of the needle while the distal wall of the needle guard catches the tip of the needle. The crimp prevents the retracted needle from retracting from the proximal hole. Additionally, the distal end section can further prevent the needle from moving forward as it blocks a direct sight path from the proximal hole to the distal wall.

With the needle guard in the unbiased position, the catheter hub is uncoupled, and is slidably movable relative to the needle guard and the safety shield housing. This allows for separation of the catheter hub from the safety shield housing. Accordingly, the catheter hub can be in a ready to use configuration.

Although the needle guard is shown as being assembled with the safety shield housing, it is also understood that the needle guard can be used with a catheter assembly without a safety shield housing. The needle guard can be applied in a suitably sized and shaped catheter hub, or needle hub without the safety shield housing. For example, such a catheter 102 can have an interior catch for coupling with the needle guard.

Aspects of the present disclosure include a catheter assembly having a catheter hub having a catheter; a safety shield or needle guard; a safety shield housing; and a needle hub having a needle; wherein the safety shield housing is disposed between the catheter hub and the needle hub; wherein the safety shield is configured to couple with a proximal portion of the catheter hub. The safety shield can be biased by the needle to then couple to the proximal portion of the catheter hub.

The present disclosure provides for a catheter assembly having a catheter hub having a catheter; a safety shield or needle guard; a safety shield housing; and a needle hub having a needle; wherein the safety shield housing is disposed between the catheter hub and the needle hub; wherein the safety shield comprises a hook housed inside the safety shield housing; wherein the hook latches onto a proximal portion of the catheter hub.

Methods of making catheter assemblies and of using the catheter assemblies as described herein are within the scope of the present invention. The methods are understood to include the use of needle guards and safety shield housings with an integrated IVC, which has a side fluid port extending from a catheter hub with a tubing and a fluid adapter at the end of the tubing.

### BRIEF DESCRIPTION OF THE DRAWINGS

These and other features and advantages of the present devices, systems, and methods will These and other features and advantages of the present devices, systems, and methods will become appreciated as the same becomes better understood with reference to the specification, claims and appended drawings wherein:

FIG. 1 shows an exploded perspective view of a needle assembly.

FIG. 1A shows a needle assembly that is an integrated intravenous catheter assembly.

FIG. 2 shows a cross sectional side view of one embodiment of a valved catheter assembly in a ready position;

FIG. 3 shows a cross sectional side view of one embodiment of a valved catheter assembly with the valve in an open position;

FIG. 4 shows an exploded view of an embodiment of a catheter assembly of the present disclosure.

FIG. 5 shows a plan top view of the catheter assembly of FIG. 4 is an assembled state.

FIG. 6 shows a perspective view of the assembled catheter assembly of FIG. 4.

FIG. 7 shows an exemplary embodiment of a safety shield housing configured to house a safety shield.

FIG. 8 shows a safety shield configured to fit with the safety shield housing.

FIG. 9 shows a cross-sectional isometric view of the catheter assembly from the proximal side in an un-activated position.

FIG. 10 shows a cross-sectional isometric view of the catheter assembly from the proximal side in an activated position.

FIG. 11 shows a cross-sectional plan side view of the catheter assembly in an un-activated position.

FIG. 12 shows a cross-sectional plan side view of the catheter assembly in an activated position.

FIGS. 13A and 13B show perspective views of an embodiment where the cover and the housing are coupled by a latch.

FIGS. 14A and 14B show an embodiment where the cover and the housing are coupled by a latch that is a living hinge.

FIG. 15 shows an embodiment where the cover and the housing are coupled by a latch coupling a proximal side of the cover and the proximal side of the housing, or a latch coupling two housing sections

FIG. 16 illustrates an embodiment of a safety shield with a folded biasing portion on the distal wall.

FIGS. 17A and 17B show cross sectional views of an assembled catheter assembly with the safety shield housing and needle guard 132.

FIG. 18 show an embodiment of a safety shield having two resilient arms.

FIG. 19 shows an exploded view of an embodiment of a catheter assembly of the present disclosure.

FIG. 20 shows a cross-sectional isometric view of the catheter assembly.

FIGS. 21A and 21B show cross-sectional side views of the interaction between the needle, safety shield, and catheter hub in biased and unbiased positions.

FIGS. 22A and 22B show isometric views of the interaction between the needle, safety shield, and catheter hub in biased and unbiased positions.

FIGS. 23A and 23B show side views of the interaction between the needle, safety shield, and catheter hub in biased and unbiased positions.

### DETAILED DESCRIPTION

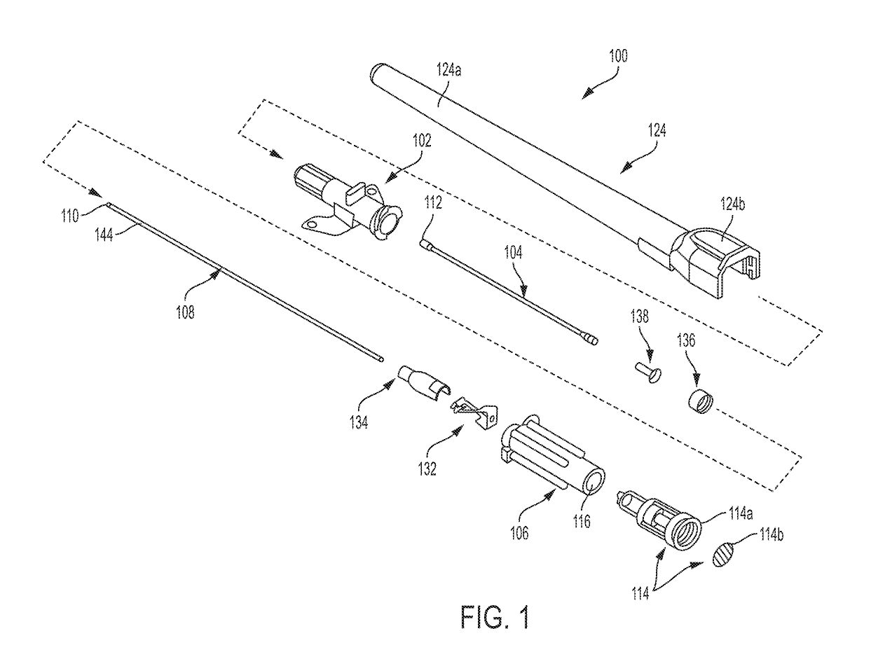

The detailed description set forth below in connection with the appended drawings is intended as a description of the presently preferred embodiments of safety shields and safety shield housings provided in accordance with aspects of the present devices, systems, and methods and is not intended to represent the only forms in which the present devices, systems, and methods may be constructed or utilized. The description sets forth the features and the steps for constructing and using the embodiments of the present devices, systems, and methods in connection with the illustrated embodiments. It is to be understood, however, that the same or equivalent functions and structures may be accomplished by different embodiments that are also intended to be encompassed within the spirit and scope of the present disclosure. As denoted elsewhere herein, like element numbers are intended to indicate like or similar elements or features. FIG. 1 illustrates details regarding components of a valved catheter assembly with a safety shield. Further information regarding various aspects valved catheter assemblies and components thereof are discussed in PCT patent applications PCT/EP2016/069619 and PCT/EP2016/069643, the contents of which are expressly incorporated herein by reference.

As shown in FIG. 1, the IV catheter assembly 100, which may more broadly be referred to as a needle assembly or a needle device, is shown comprising, a catheter hub 102 with a catheter tube 104 and a bushing 138. The bushing 138 can be configured to wedge the proximal end of the catheter tube 104 against the interior wall surfaces of the catheter hub 102 to retain the catheter tube 104 to the catheter hub 102.

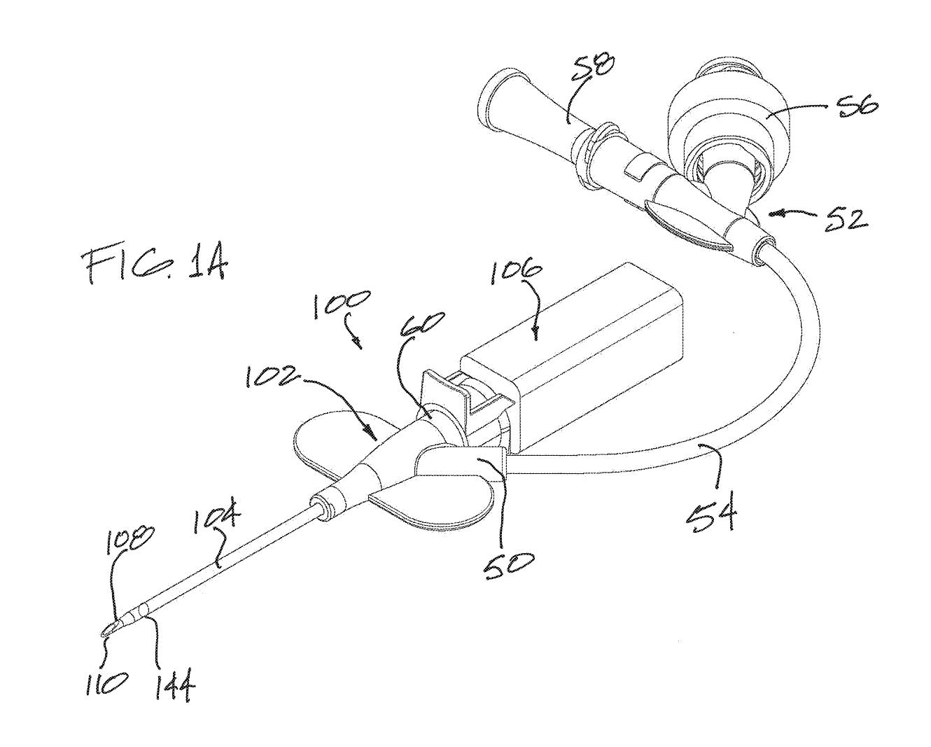

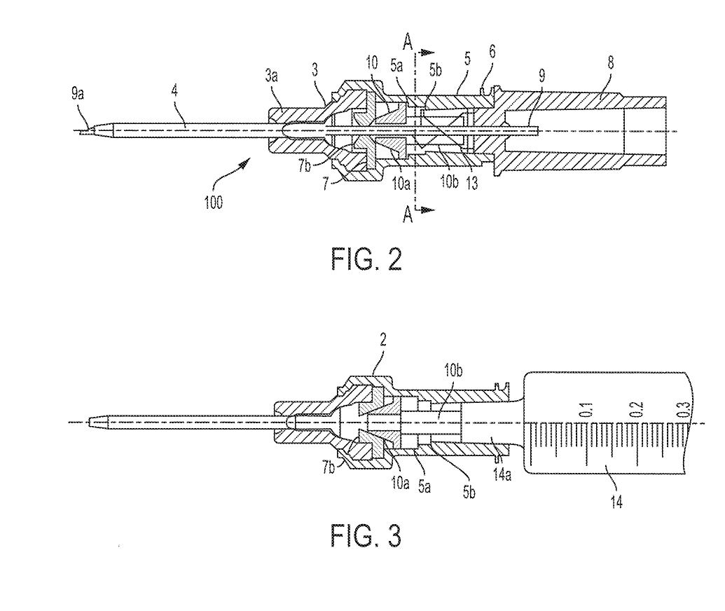

Interiorly of the catheter hub 102, a septum or valve 136, an actuator or valve opener 134 and a needle guard 132, such as a safety shield or tip protector, are provided. A needle 108, which has a change in profile 144 proximal of the needle tip 110, can be inserted through the proximal opening of the catheter hub 102 with the needle tip 110 protruding from the distal opening 112 of the catheter tube 104 in a ready to use position. The catheter hub 102 can be made from a single hub body as shown or from two or more hub bodies assembled together, as discussed below with reference to FIGS. 2 and 3. A cannula hub or needle hub 106 can attach to the proximal end of the needle 108 and can contact the proximal end of the catheter hub 102 when assembled thereto in the ready to use position. The proximal opening of the catheter hub 102 can be sized with a female Luer taper, optionally with external threads, to engage with a male Luer tip in a Luer slip or a Luer lock.

The needle guard 132, which is understood to include structural features for guarding the needle tip from unintended needle sticks, is configured to be removed with the needle 108 following successful venipuncture and the valve 136 and valve actuator 134 are configured to remain with the catheter hub 102 for controlling fluid flow therethrough. The actuator 134 is configured to be pushed distally by a male tip into the valve 136 to open the valve for fluid flow, as discussed below. Further information regarding the needle guard 132 is discussed in U.S. Pat. No. 8,568,372, the contents of which are expressly incorporated herein by reference. In alternative embodiments, the needle guard 132 can embody multiple components that cooperate to block the needle tip from unintended needle sticks. For example, the needle guard can comprise a spring loaded needle carrier having a needle attached thereto. Following successful venipuncture, a release tab can be pressed to release the spring to then move the needle carrier and needle inside a protective barrel to block the needle tip from unintended needle sticks.

A flash back plug or blood stopper assembly 114 can be connected to the needle hub 106 to stop blood flow out the flashback chamber 116 of the needle hub 106. The flash back plug 114 can be provided at the proximal end the needle hub 106 to allow air to vent but stops blood from spilling out the proximal end of the body of the flash back plug 114, which has a chamber 114a and a hydrophobic filter 114b assembled to the chamber. Alternatively, a syringe can be attached to the proximal end of the needle hub 106. A second valve 136 and actuator 134 can also be placed within the needle hub 106.

A protective cap 124 with a sleeve 124a and a saddle 124b can be provided to cover the needle 108 during packaging and before use, which is conventional. The saddle 124b can surround at least part of the catheter hub 102 and the needle hub 106 and be removably engaged to the needle hub. The cap 124 should be removed from the needle assembly before use. The catheter hub 102 can be provided with a pair of wings to facilitate securement of the catheter hub to a patient following use.

FIG. 1A shows an embodiment of a catheter assembly 100 provided in accordance with other aspects of the present invention, which is also known as an integrated intravenous catheter assembly or integrated IVC. The catheter assembly 100 comprises a catheter hub 102 and a catheter tube 104, a needle hub 106 having a needle 108 extending through the catheter tube 104, with a needle tip 110 extending distally of the catheter tube opening. A crimp or change in profile 144 formed with the needle 108 is located proximally of the needle tip and inside the catheter tube in the ready to use position.

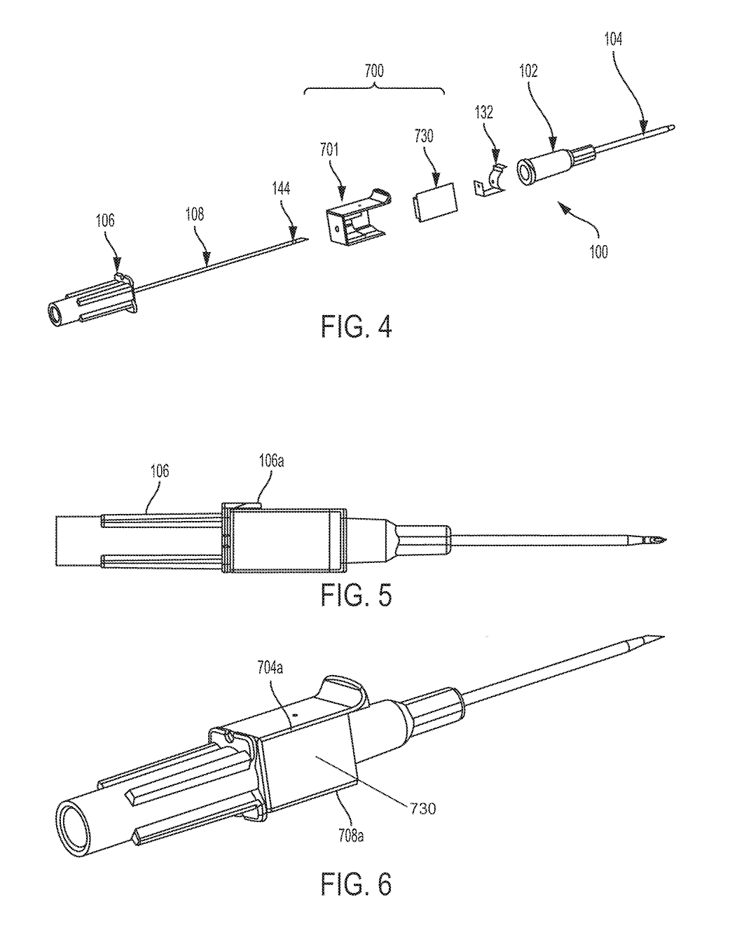

The catheter hub 102 has a side fluid port 50, and a fluid adaptor 52 attached to the fluid port 50 by a tubing 54 having a lumen for fluid flow between the port 50 and the adaptor 52. As shown, the fluid adaptor 52 is a Y-site comprising at least one needleless female Luer connector 56. The other opening of the Y-site can have a conventional vent plug 58. The proximal end 60 of the catheter hub 102 can be equipped with a septum, a seal or a valve and prevents flow thereacross after removal of the needle 108 and the needle hub 106 following successful venipuncture. A needle shield or guard 132 can be incorporated between the needle hub 106 and the catheter hub 102. The needle guard 132 can be placed inside a safety shield housing 700 (FIGS. 4, 7, 21A and elsewhere) and the combination needle guard and safety shield housing can be placed between the needle hub and the catheter hub, as further discussed below. The catheter hub 102 is shown with a pair of wings. The needle hub can alternatively have a wing that extends distally along a side of the catheter hub opposite the side fluid port 184, instead of the wing on that side.

Following successful venipuncture, the needle 108 and needle hub 106 are removed from the catheter hub and the catheter tube and the needle tip 110 is covered or protected by the needle guard 132, and optionally with the safety shield housing 700. Blood flow is stopped by a seal or septum located at the proximal end of the catheter hub, proximal of where the side fluid port 50 opens into the catheter hub so as to not interfere with fluid flow through the side fluid port. IV fluid or other medicaments can flow into the catheter hub via connection to the fluid adapter 52.

FIGS. 2 and 3 show the IV catheter assembly 100 in a first state with the safety shield in place in a ready to use position in which the needle tip extends out the distal opening of the catheter tube and a second state where the needle hub is removed from the catheter hub following use. After removal of the needle hub, a male Luer tip, such as a syringe tip, can be inserted into the proximal opening of the catheter hub to push open the valve with the valve opener.

FIG. 2 shows an embodiment of an IV catheter assembly 100 with a valve 7 having a catheter hub 2, which in the embodiment shown has a two-part hub body. Further information regarding the catheter assembly 100 having the valve is discussed in U.S. Pat. No. 9,114,231, the contents of which are expressly incorporated herein by reference. In other examples, a ported catheter assembly or an integrated catheter assembly having an integrated tubing can be practiced with the valve and valve opener described herein.

A distal hub element or first hub part 3 of the catheter hub has a holding section 3a, such as a nose section, in which a catheter tube 4 is held, such as with a metal bushing. The proximal end of the first hub element or part 3 has an enlarged diameter relative to the nose section of the distal end portion and forms a connecting section for connecting with a rear hub element or second hub part 5. The two-part hub configuration can facilitate assembly of a valve and a valve opener, as further discussed below. However, catheter assemblies described herein are not limited to a two-part hub as a singularly formed hub or three or more hub parts may be used to form a catheter hub without deviating from the scope of the catheter assemblies described herein.

The distal end of the rear hub part 5 overlaps the proximal end of the front or first hub part 3 and which is provided at its proximal end with a female Luer fitting with exterior threads 6, forming a female threaded Luer. In some examples, the threads can be omitted and the proximal opening can function as a female Luer slip. Between the two hub parts 3 and 5, a valve 7 in the form of a check valve shaped as a cylinder with a valve disk, valve disc, or a valve disk without a skirt is inserted and is fixed in place by the two hub elements 3 and 5. In other embodiments, a single hub body is used, such as hub 3 only, and the valve 7 is held in place by placing the valve next to or against a shoulder in the interior of the hub element 3. A second shoulder may be incorporated to secure the valve in place with adhesive or bonding being optional to further assist holding the valve in place. In still yet other examples, a single catheter hub body is used with internal shoulders or undercuts incorporated for retaining the valve 7 therein, without adhesive or bonding. In still another embodiment of the present disclosure, the valve is held in the catheter hub by an interference fit.

In the ready position of FIG. 2, a nose section of a needle hub 8 is inserted into the catheter hub 2. A hollow needle 9 is fixed to the nose section of the needle hub and extends through the valve 7, the catheter hub 2, and the catheter tube 4 so that the needle tip 9a is exposed beyond the tapered end of the catheter tube 4. Between the needle hub 8 and the valve 7 and inside the catheter hub 2, a valve opening device, valve actuator, or valve opener 10 with a nose end 10a for opening the valve 7 is slidably or displaceably arranged, as shown in FIG. 3, which is shown pushed in a distal direction by a syringe 14. In an example, the nose end 10a of the valve opener has a truncated cone-shaped locating section or a tapered section. In other examples, the valve actuator 10 has a nose section with a radiused tip, a square tip, or a tip with angular surfaces.

On the proximal side of the valve actuator 10, a plunger section or pusher end 10b is provided. The plunger section is sized and shaped to be pushed by the male Luer conical fitting of a medical implement, such as a syringe tip or an IV tubing adaptor, to open the valve 7. The pusher end 10b adjoins the nose end 10a. The pusher end 10b can have a surface to be pushed. The pusher end 10b can have one or more gaps for fluid flow thereacross. In an example, the nose section 10a incorporates a groove to facilitate engagement with the valve so that even if the medical implement no longer pushes on the valve actuator 10, the nose section remains engaged with the valve to keep the valve in an open position for fluid flow.

As explained above, a typical exchange of elements could be the use of the valve opener 10 in the embodiment of FIGS. 2 and 3. In one example, there is only a single element or leg forming the pusher end. In another example, two legs with a hollow space therebetween or a gap are provided. The two proximal ends of the two legs can provide a surface to be pushed by the medical implement. The space between the two legs can be sized to receive a safety shield, or needle guard 13. For example, the safety shield 13 can be positioned in the space between the two legs. In still other examples, the pusher end is a cylinder having one or more openings through the cylinder to provide space for engagement with a needle guard, which can engage the catheter hub through the one or more openings or engage an edge of each of the openings of the cylinder, if more than one opening.

In other examples, a third housing having a cavity is positioned between the catheter hub 2 and the needle hub. The safety shield element 13 may be positioned in the cavity of the third housing and the third housing having mechanical features to engage the catheter hub and the needle hub. For example, the third housing can have an extension that contacts the interior or the exterior of the catheter hub to secure the third housing to the catheter hub and/or to prevent early activation of the needle guard. In some examples, the needle guard is omitted and the catheter assemblies only incorporate a valve and a valve opener. In still other examples, the valve opener can be omitted and the valve is sized and shaped to open or close based on fluid pressure.

On withdrawal of the hollow needle 9 from the catheter hub 2 following successful venipuncture, a change in profile 144 provided near the needle tip 9a and having the form of a radial projection on the hollow needle, such as by crimping, engages with a perimeter defining the proximal opening on the rear wall or proximal wall 13c of the safety shield 13 so that the safety shield 13 can be removed from the catheter hub with the needle 9. As the needle tip moves proximal of the two distal walls of the two arms, the two arms move, such as spring or deflect radially to disengage from the interior of the catheter hub. As the arms 13a and 13b of the safety shield move radially, the arms, or the distal walls of the arms, cover the needle tip to prevent unintended needle sticks. In other examples, the change in profile can include a sleeve, a notch, or a material buildup on the shaft of the needle.

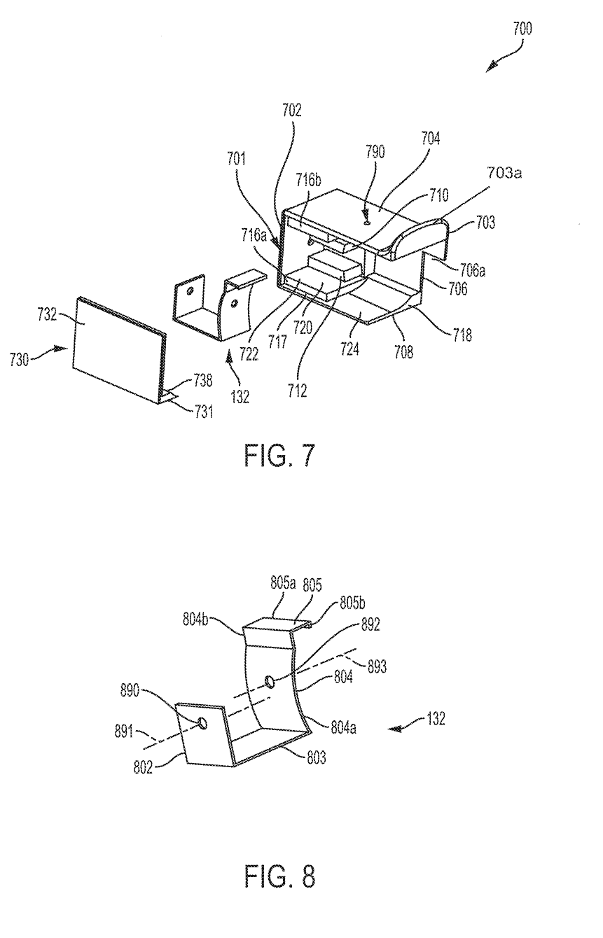

Subsequent to removal of the needle following successful venipuncture, the two or more flaps of the valve 7, due to their elastic properties, close the one or more slits through the depth of the valve disk or disc so that no blood or substantially no blood can flow out through the catheter 4. As further discussed below with reference to FIGS. 6 and 7, valves provided herein can include three slits 54 starting from the middle of the valve and extending radially over a short radial distance towards the outer perimeter to form elastic flaps that can be expanded by the hollow needle and closed when the needle is removed. In some examples, the flaps can remain open and engaged with a valve opener, which can be referred to as a one-time use valve. The number of slits defining the number of flaps can vary, ranging from one slit to more than four slits.

FIG. 3 shows the insertion of a syringe 14 into the proximal opening of the catheter hub 2 to either inject a fluid, such as medicament, through the catheter hub or draw a sample of a bodily fluid like blood. As shown, the syringe tip 14a of the syringe 14 abuts the pusher end 10b of the valve actuating element 10 and pushes it against the valve 7 so that the nose section 10a of the valve opener advances against the flaps of the valve to open the slits 7a thereby opening the valve so that liquid can flow there-across.

As shown, the nose end 10a of the actuator 10 is inclined. Thus, as the syringe is removed and the forward force on the actuator is removed, the elasticity of the material of the valve 7 is sufficient for the two or more flaps to uncoil and push the actuator in the proximal direction to close the seal 7. The valve 7 therefore automatically closes upon withdraw of the pushing force on the actuator and can be re-used by inserting a male Luer tip into the catheter hub to again advance the valve opener into the valve.

A shoulder 5a is shown in the catheter hub 5 in FIGS. 2 and 3. The shoulder 5a acts as a proximal stop for the actuator 10 when the flange on the actuator abuts the shoulder, which defines the proximal most position of the flange of the actuator. In other examples, the second hub section may incorporate other structural features, such as a tapered internal cavity, to stop the proximal travel of the actuator.

Also shown in the inner circumference of the bore of the hub element 5 is a second shoulder 5b just proximal of the first shoulder 5a. The two radially outer areas of the spring arms of the needle guard, which may be referred to as elbows between the distal walls and the two elongated sections of the two arms, are configured to abut the second shoulder 5b in the ready position in FIG. 2. When the needle hub 8 with the hollow needle 9 is removed from the catheter hub 2, the safety shield 13 is held generally stationary by the shoulder 5b until the change in profile, such as a crimp, near the needle tip comes to abut on the rear proximal wall of the needle guard and the needle tip moves proximally of the two distal walls on the safety shield 13. At this point, the two spring arms, which are no longer restrained in the radial direction by the needle, spring inwards to cover the needle tip and separate from the second shoulder 5b, whereupon the safety shield 13 with the hollow needle 9 can be removed from the catheter hub. Further information regarding the safety shield 13 is discussed in U.S. Pat. No. 7,736,339, the contents of which are expressly incorporated herein by reference.

FIG. 4 illustrates an exploded view of an embodiment of a catheter assembly 100 of the present disclosure. Additional components not illustrated in FIG. 4 but understood from the incorporated references can be added to the catheter assembly 100 or substituted with other components as desired. In comparison to FIG. 1, the catheter assembly 100 of the present embodiment includes an additional safety shield housing 700 between the catheter hub 102 and the needle hub 106. Further, while the catheter hub 102 shown in FIG. 4, and elsewhere, illustrates a standard straight catheter hub, the catheter hub can be an integrated catheter hub as shown in FIG. 1A, which has a side fluid port 50, a tubing 54, and a fluid adapter 52.

The catheter assembly 100 generally comprises a catheter tube or, its shorthand version, a catheter 104 and a catheter hub 102 at a distal end of the catheter assembly 100. A distal end of the safety shield housing 700, which comprises a housing structure 701 and a cover 730, can contact a proximal end of the catheter hub 102. The safety shield housing 700 can house the needle guard 132. For example, the needle guard can be spaced from the catheter hub when using the safety shield housing 700. This is in contrast with the embodiment illustrated in FIG. 1 wherein the needle guard 132 is housed in the catheter hub 102. In this way, the catheter hub does not have the design constraints of having to be sized and shaped to receive and house a safety shield. The needle hub or cannula hub 106 can attach to a proximal end of the needle 108 and can contact a proximal end of the safety shield housing 700 when assembled thereto in the ready to use position.

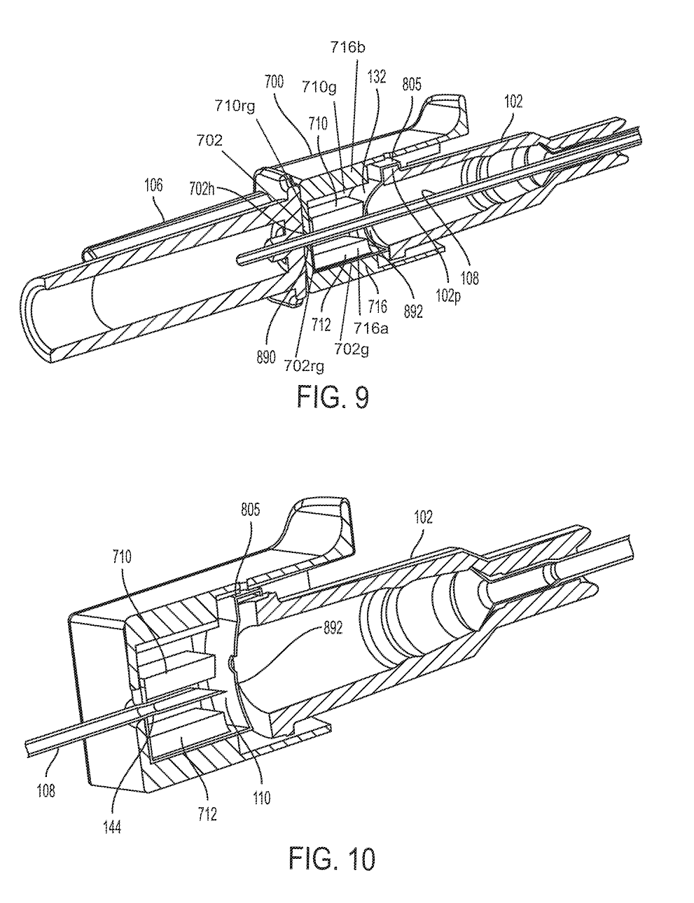

The change in profile 144 of the needle 108 can be configured to engage with an outer circumference defining a proximal opening on the rear wall or proximal wall of the needle guard 132 so that the needle guard 132, and hence the safety shield housing 700, can be removed from the catheter hub 102 with the needle 108. The proximal wall of the needle guard 132 is slidably located between the location of the change in profile 144 and the proximal end of the needle 108. Thereby, the proximal wall of the needle guard 132 can be prevented from passing the change in profile 144 and from becoming separated from the needle 108. Further explanation of the interaction between the needle 108 and the needle guard 132 is discussed below with respect to FIGS. 9-12.

As described above, embodiments of the components from the incorporated references can be implemented for the various components of the catheter assemblies discussed herein. For example, regarding the catheter hub, it can be a wingless catheter hub, a winged catheter hub, a ported catheter hub, or a catheter adapter with an inner lumen with a valve or permanent septum.

FIGS. 5 and 6 illustrate different views of the catheter assembly 100. FIG. 5 illustrates a top view of the catheter assembly 100. The needle hub 106 can have an orientation guide 106a. The orientation guide 106a can embody a tab and extend from a distal end of the needle hub 106 in a distal direction to provide a surface for the safety shield housing 700 to align therewith. The orientation guide 106a can be a substantially rectangular feature for contacting a side of the safety shield housing 700. In some examples, the safety shield housing can have a recess or channel to receive the orientation guide 106a, which can seat flushed within the recess or project above the recess.

The orientation guide 106a can ensure proper orientation of the needle 108 relative to the safety shield housing 700. This can ensure that the safety shield housing 700 is oriented upward such that a distal wall 703 can be properly oriented. Additionally, this can ensure access to a hope 790 on the safety shield housing 700. Further, the safety shield housing 700 can then orientate relative to the catheter hub, thus indirectly the needle, especially the needle bevel at the needle tip, can orientate relative to the catheter hub. As shown in FIG. 6, the bevel preferably faces up, away from the patient, when in the ready to use position.

FIG. 5 illustrates a plan top view of the catheter assembly 100. As shown and as further described below, the assembled catheter assembly 100 allows for the catheter hub 102 to be partially retained inside an interior of the safety shield housing 700 in ready to use position.

FIG. 6 illustrates a perspective view from the proximal top side of the assembled catheter assembly 100. In the viewing angle shown, the cover 730 can be inset to be flush with the edges 704a, 708a of the sides of the safety shield housing 700. The cover 730 can snap fit into the side opening of the housing 500. Optionally, the cover 730 can be glued to the safety shield housing 700.

FIGS. 7 and 8 further illustrate an exemplary embodiment of the safety shield housing 700 as shown in FIGS. 4-6.

FIG. 7 illustrates an exemplary embodiment of the safety shield housing 700 configured to house a needle guard 132. The safety shield housing 700 can have two parts or can comprise two parts, which can include a housing structure 701 and a cover 730. The housing structure 701 can have a number of distinct sides. In an example, the distinct sides can include a proximal side 702, a distal side 703, a top side 704, a lateral side 706, and a bottom side 708. The top side 704, the lateral side 706, and the bottom side 708 define a channel or recess that can constitute an interior 720 of the safety shield housing 700. With the proximal side 702 and the channel or recess, a cavity is defined for accommodating a needle guard 132. The open side of the channel or recess, or the side opposite the lateral side 706, can be configured to be coupled with the cover 730 or to receive the cover.

The exterior surfaces of the proximal side 702, the top side 704, the lateral side 706, and the bottom side 708 can each be substantially flat. Alternatively, one or more of the surfaces can have curvature or another geometric shape. In embodiments, the housing structure 701 can have a cylindrical exterior. Optionally, surface features can be provided on the exterior of the housing structure 701, such as bumps or projections for gripping purposes.

The distal side 703 can extend from the top side 704. In an example, the distal side can extend outwardly from the housing structure 701. The distal side 703 can have a fillet bending radially of the surface of the top side 704. The distal side 703 can act as a stop or a finger tab for a user's finger to push against for leverage when using the needle device. The distal side 703 has an outer edge or free end 703a that points away from the needle when the housing structure 701 is mounted onto the needle.

The top side 702 can further comprise a hole 790 located at an intermediary location of the top side 702. The hole 790 can be used to position a needle guard 132, as described below and with reference to FIGS. 9-12. The hole 790 can be sized to accommodate a desired safety shield or needle guard setting tool to provide access to the interior of the safety shield housing 700 for manipulating the needle guard 132 with the setting tool when installing the needle guard in the safety shield housing 700, as further discussed below.

The lateral side 706 can have a notch portion 706a at a distal end. The notch portion 706a can be viewed as a cut-out on the wall surface of the lateral side 706. The notch portion 706a can have two edges having a generally L-shape, which forms the cut-out on the wall surface of the lateral side. Part of the lateral side 706 without the cut-out can extend approximately to the distal side 703 extending from the top side 704. However, the part of the lateral side 706 having the notch portion 706a can be shorter and can define an axially shorter portion or wall surface of the lateral side 706. In an example, the bottom side 708 can extend evenly with the axially shorter portion of the lateral side.

In the interior 720 of the housing structure 701, there can be a proximal region 722 and a distal region 724. The proximal region 722 can have stop pieces 716a, 716b. The stop pieces 716a, 716b may embody plates or bands forming thicker portions of the bottom side 708 and top side 704, respectively, that can act as an insertion limiter for the catheter hub 102 when the catheter hub 102 is inserted from a distal end of the safety shield housing 700 in an assembled or ready position. The stop pieces 716a, 716b can be generally rectangular materials or pieces extending inwardly from the top side 704 and the bottom side 708 but short of the distal ends or distal edges of the stop side 704 and the bottom side 708. Thus, the top side 704 and the bottom side 708 each comprises a section of greater thickness at the proximal region 722, due to the stop pieces, than at the distal region 724. The stop pieces can be unitarily formed with the top side 704 and the bottom side, such as by plastic injection molding.

Surfaces of the stop pieces 716a, 716b may be generally parallel to the top side 704 and the bottom side 708. The stop pieces 716a, 716b can be inset by a distance 717 from at least one edge of the top side 704 and the bottom side 708 of the housing structure 701. In an example, both the top side and the bottom side have an inset so that the housing structure 701 is said to have two insets 717. As shown, the insets 717 are provided along the open side of the housing structure for receiving the cover 730. The inset can be provided to aid in configuration for coupling of the cover 730, as described below, so that the cover 730 seats flushed with the edges of the housing structure 701.

The distal region 724 can have a catheter ramp 718 on the bottom side 708. The catheter ramp 718 can extend from a distal end of the housing structure 701 to one of the stop pieces 716a, 716b. The catheter ramp 718 can be configured to aid in guiding the catheter hub 102 in axial movement. The top side 704 can also have a catheter ramp 718. In the illustrated embodiment, part or all of the catheter ramp 718 can have a surface with an arcuate section or shape that can correspond to an arcuate guide for a cylindrical catheter hub 102 as shown in FIG. 4. The catheter ramp 718 can be shaped to correspond to an exterior shape of the catheter hub 102.

The interior 720 of the housing structure 701 is configured to accommodate a needle guard 132, described below with reference to FIG. 8.

The housing structure 701 can be configured to fit with the cover 730 or to receive the cover such that the cover 730 covers the open side of the housing structure to cover the interior 720. The cover 730 can fit on a side of the housing opposite of the lateral side 706 of the housing structure 701 so that in the assembled state, as shown with reference to FIG. 6, the edges of the cover 730 seat within the insets 717 in a flushed configuration.

The cover 730 can have a cover side 732 and a fitment tab 731. The fitment tab 731 can include a catheter ramp 738, which can have a surface with an arcuate section to provide an arcuate guide for a cylindrical catheter hub 102, as shown in FIG. 4 and similar to the catheter ramp 718 on the lateral side 706. The fitment tab 731 can extend a length that is shorter than the width of the cover 730. For example, the fitment tab 731 can extend from a distal portion of the cover 730 while leaving the proximal portion of the cover on the side of the fitment tab free or open of any tab. The catheter ramp 738 can be shaped to correspond to an exterior shape of the catheter hub 102. The fitment tab 731 can be configured to fit into the open distal region 724 of the bottom side 708 distal of the stop piece 716a and the thickness of the cover 730, such as the edges of the cover, can fit into the inset 717 adjacent to the stop pieces 716a, 716b.

The cover side 732 of the cover 730 can fit between the top side 704 and the bottom side 708 of the housing structure 701 and forming one of the sides thereof. Alternatively, the cover side 732 can abut the side edges of the top side 704 and the bottom side 708. The cover 730 can have a length that matches the bottom side 708, and not fit within the insets 717. Alternatively, the cover 730 can be shorter or longer than the housing structure 701.

FIG. 8 illustrates a needle guard 132 configured to fit with the safety shield housing 700. The needle guard 132 can have a proximal wall 802, an intermediary wall 803, a distal wall 804, and a hook 805. The needle guard 132 can be unitarily formed from a stamped metal piece and then manipulated or cold worked to a final shape. In an example, the metal is stainless steel and the intermediary wall 803 and/or distal wall 804 can act as resilient arms, spring arms, or resilient springs, such as cantilever springs. In other examples, the needle guard 132 can be made from two or more pieces that are assembled or welded together.

An end of the proximal wall 802 can join with an end of the intermediary wall 803. Another end of the intermediary wall 803 can join with an end of the distal wall 804. Another end of the distal wall 804 can join with a hook 805. The proximal wall 802 can be substantially perpendicular to the intermediary wall 803 with some angular variation being optional. With reference to FIGS. 9-12, the proximal wall 802 can be configured to be substantially perpendicular to a longitudinal axis of the needle 108 when the needle guard 132 is mounted to the safety shield housing 700 and the combination mounted onto the needle.

At intermediary locations of the proximal wall 802 and the distal wall 804, there can be perimeters defining a proximal hole 890 and a distal hole 892, respectively. The proximal hole 890 and the distal hole 892 can have diameters that are slightly larger than an outer diameter of the needle configured to pass through the proximal hole 890 and the distal hole 892. In an example, the proximal and distal holes can have a half to three thousandths total clearance with the needle diameter with a different range contemplated. In other examples, the distal hole 892 can be larger than the proximal hole 890 and the distal hole can allow a needle crimp to pass thereacross, as further discussed below. The holes can be circular or round or can have a shape that is other than round, such as polygonal.

The proximal hole 890 and the distal hole 892 can be arranged such that in a relaxed state, such as an unbiased state of the needle guard 132, a longitudinal axis 891 of the proximal hole 890 is offset from a longitudinal axis 893 of the distal hole 892. For example, elevation-wise from the surface of the intermediary wall 803, the center of the distal hole 892 can be higher than the center of the proximal hole 890. The proximal hole 890 and the distal hole 892 can be arranged such that when the needle guard 132 is in a biased, or compressed state, the proximal hole 890 and the distal hole 892 can be aligned. In an example, the centers of the proximal and distal holes are about at the same height, elevation-wise, from the intermediary wall when the needle guard is in the biased state. Viewed differently, the longitudinal axis 891 of the proximal hole 890 can align with the longitudinal axis 893 of the distal hole 892 when the needle guard is in the biased state.

In an example, the proximal hole 890 can be sized and shaped to have a dimension or a diameter sufficiently small to catch a stop or change in profile 144, such as a crimp or a weld bead, on the needle during withdrawal of the needle following venipuncture. This relative sizes of the change in profile 144 and the proximal hole opening can create an interference and can prevent the needle guard 132 from separating distally off of the needle 108 when the needle hub 106 is withdrawn from the catheter hub 102, as shown in FIGS. 9-12. Alternatively, the proximal hole can have an extended emboss configured to catch the change in profile 144, or crimp, on the needle.

The distal wall 804 of the of the needle guard 132 can be sized and shaped to flex between a biased position and an unbiased position. The distal wall 804 can have the ability to flex such that the end of the distal wall 804 joined with the hook 805 can be deflected with movement along a plane substantially perpendicular to the longitudinal axis of the needle.

The distal wall 804 can be formed with a curve shaped portion 804a, whereby the curve shape allows for elastic buckling spring effect to occur with the distal wall 804. The curve shaped portion 804a can join with the intermediary wall 803 with an acute angle. The distal hole 892 can be located on the curve shaped portion 804a. The curve shaped portion 804a can join at another end with a flat portion 804b. The flat portion 804b can be substantially perpendicular to the longitudinal axis of the needle 108. The distal wall 804 can include both the curve shaped portion 804a and the flat portion 804b. The flat portion 804b can join with the hook 805. In an example, the curve shaped portion 804a has a larger surface area than the area of the flat portion 804b. In other examples, the relative surface area sizes of the two portions 804a, 804b are reversed.

The hook 805 can be composed of a first portion 805a and a hooking portion 805b. The hooking portion can be a lip or a tab for engaging the catheter hub. The hook can alternatively have a curved surface or an arcuate surface for engaging the catheter hub. Optionally, the hook can have at least one of a lip and a curved surface. The hook 805 can be configured to allow for hooking or latching on to a Luer thread on a catheter hub or a corresponding detent formed on the catheter hub. Such a detent formed on the catheter hub can take the form of a flange, Luer locking ears, a notch, a slot or a peg.

As illustrated in FIG. 8, the first portion 805a can be configured to extend in a direction towards a distal end of the catheter hub in order for the hooking portion 805b to clear the corresponding Luer thread or detent of the catheter hub for hooking.

The hook 805 can have different geometries as desired and necessary for interfacing with a corresponding catheter hub. In embodiments, the hook 805 can be triangular in shape with an upward angled first portion 805a and a downward angled hooking portion 805b. In embodiments, the hook 805 can have a hooking portion 805b without a first portion 805a. For example, the hook 805 can be comprised of an arcuate portion or a curved portion.

The needle guard or safety shield 132 can be made of thin metal sheet being stamped into the desired shape and then cold work or formed, such as bent, into final shape wherein, in the biased position, the safety shield engages the catheter hub and in the unbiased position the safety shield disengages the catheter hub as described below with reference to FIGS. 9-12.

Although the needle guard 132 is shown as being assembled with the safety shield housing 700, it is also understood that the needle guard 132 of FIG. 8 can be assembled with a catheter assembly 100 without a safety shield housing 700. For example, the needle guard 132 can be applied in a suitably sized and shaped catheter hub 102 with a Luer taper, similar to that of FIG. 1, or needle hub 106 without the safety shield housing 700. For example, such a catheter hub 102 can have an interior catch for coupling with the needle guard 132.

FIGS. 9-12 illustrate cross-sectional views of an assembled catheter assembly with the safety shield housing 700 and needle guard 132 illustrated in FIGS. 7 and 8.

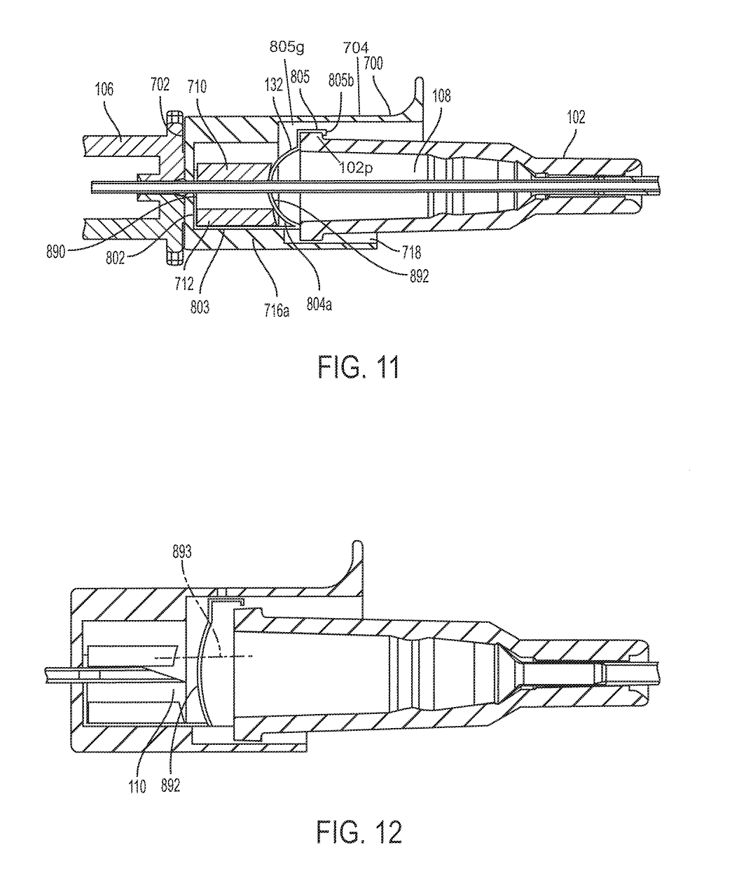

FIGS. 9 and 10 illustrate isometric cross-sectional views of the catheter assembly from the proximal side. FIGS. 11 and 12 illustrate cross-sectional side plan views of the catheter assembly.

FIGS. 9 and 11 show the catheter assembly in a first position or ready state, such as a ready to use state or position, with the safety shield in a biased, un-activated position. FIG. 11 shows the needle guard 132 retained in a biased position by the needle 108 extending through the proximal hole 890 and the distal hole 892. In the un-activated position, the hook 805 couples to a latching portion 102p of the catheter hub 102, thereby preventing premature activation of the needle guard 132. The engagement between the hook 805 and the latching portion 102p also allows the catheter hub 102 and the safety shield housing 700 to remain together until separation of the needle guard from the catheter hub.

As shown in FIG. 9, the first or upper ramp 710 is spaced from the stop piece 716b at the top side 704 by a slot or gap 710g. The first ramp 710 is also spaced from the proximal side 702 of the housing structure 701 by a rear slot or gap 710rg. For example, the upper ramp 710 can attach to the lateral side 706 and can extend from the lateral side 706 towards the open side of the housing structure 701 and can be spaced from the stop piece 716b and the proximal side 702 by gaps 710g and 710rg.

Similarly, the second or bottom ramp 712 can attach to the lateral side 706 and can extend from the lateral side 706 towards the open side of the housing structure 701. The second ramp 712 can be spaced from the stop piece 716 at the bottom side 708 by a slot or gap 702g and spaced from the proximal side 702 by a rear slot or gap 702rg. As, further discussed below, the gap 702g between the second ramp 712 and the bottom side 708 is configured to receive the intermediate wall 803 of the needle guard 132 when mounting the needle guard to the housing structure 701. The proximal wall 802 of the needle guard 132 is configured to fit into the rear gaps 702rg, 710rg adjacent the proximal side 702.

The ramps 710, 712 of the housing can act as guides for the needle 108 by maintaining the needle therebetween and also provide a slot or gap 702g for the placement of the needle guard 132. The intermediary wall 803 of the needle guard 132 can be positioned between the bottom ramp 712 and one of the stop pieces 716a. The proximal wall 802 of the needle guard 132 can be positioned between the top and bottom ramps 710, 712 and the proximal side 702 of the safety shield housing 700, at rear gaps 702rg and 710rg. The proximal wall 802 and the intermediary wall 803 of the needle guard 132 can be firmly fitted in the slots between the ramps 710, 712, proximal side 702, and the stop piece 716a. The needle guard can be said to comprise a first part and a second part and wherein the first part of the needle guard is located in a slot between a ramp and a bottom side of the safety shield housing and the second part of the needle guard is located in a slot between the ramp and the proximal side of the safety shield housing. In an example, the dimension of the rear gap 702rg between the second ramp 712 and the proximal side 702 is sized to fit the proximal wall 802 of the needle guard to hold the needle guard securely in place. In an example, the dimension of the gap 702g between the second ramp 712 and the stop piece 716a is also sized to fit the proximal wall 802 of the needle guard to hold the needle guard securely in place. The proximal wall 802 can be held in a tight fit between the second ramp 712 and the proximal side 702 and between the second ramp 712 and the stop piece 716a.

The size, shape, and location of first, upper, or top ramp 710 of the housing can also prevent the sharp distal tip 110 of the needle 108, shown in FIGS. 10 and 12, from pivoting a sufficient amount to be able to be tilted upwards towards the distal hole 892 on the distal wall 804 (FIG. 8) of the needle guard 132 in an unbiased position of the needle guard to prevent re-exposing the needle tip through the distal hole 892.

The first and second ramps 710, 712 can extend distally forward in the safety shield housing 700 such that the ramps contact the distal wall 804 of the safety shield in a biased position, such as shown in FIG. 11. The ramps 710, 712 can act as a hard stop for compression or biasing of the needle guard 132. In other examples, the ramps can extend short of the positions shown in FIG. 11 and not touch the distal wall 804 in the safety shield biased position.

Alternatively, the ramps 710, 712 can be formed and/or molded on the cover 730 instead of the housing structure 701. Said differently, in an alternative embodiment, the ramps 710, 712 can separate from the housing structure 701. The ramps can be placed into the interior 720 of the housing structure 701 when mounting the cover 730 to the open side of the housing structure 701.

In an example, the ramps 710, 712 can be integrally molded as a single component together with the housing structure 701 of the safety shield housing 700. The ramps 710, 712 can be molded in a separate step from molding either the housing structure 701 or the cover 730. The two ramps can be similar in size and shape or can be different.

The proximal wall 802 and the intermediary wall 803 of the needle guard can be substantially perpendicular to one another. In this way, the needle guard 132 can be retained in the safety shield housing 700. In other examples, the proximal wall 802 and the intermediary wall 803 of the needle guard are located less than or greater than 90 degrees from one another to thereby create a biasing effect against the structures of the safety shield housing 700 when mounted within the gaps 702g, 702rg, 710rg. The intermediary wall 803 can be retained in the slot or gap 702g between the bottom ramp 712 and one of the stop pieces 716a. The material thickness of the needle guard 132 can fit into the slots or gaps 702g, 702rg, 710rg in a size-on-size fit or with a slight clearance that enables some movement or shifting of the needle guard relative to the housing structure.

The distal wall 804 of the needle guard 132 can be positioned in the distal region 724 of the safety shield housing 700, such that the distal wall 804 can move between a biased state and an unbiased state. The top ramp 710 and the bottom ramp 712 can have distal ends 710a, 712a that have tapered distal end surfaces configured to approximately match the contour of the curved shape portion 804a of the needle guard 132 in the biased state of the needle guard. Optionally, the distal end surfaces of the top and bottom ramps 710, 712 are not tapered.

In an un-activated, biased position, as shown in FIGS. 9 and 11, the curve shaped portion 804a of the needle guard 132 is constrained by the needle 108. That is, when the needle shaft passes through the proximal hole 890 on the proximal wall 802 and the distal hole 892 on the distal wall 804, the needle constrains the distal wall against the intermediary wall 803 are create a biasing or resilient force between the distal wall and the needle. In this way, the curve shaped portion 804a is flexed, or biased, such that the distance from the hook 805 to the intermediary wall 803 is shorter than when the needle guard 132 is unbiased by the needle.

As shown, a hole 790 for accessing the interior 720 of the safety shield housing 700 with a tool can be positioned above the hook 805 to facilitate assembly of the needle through the distal hole 892. When the access hole 790 is so positioned, a tool can be inserted into the hole 790 at the top side 704 of the housing structure 701 to bias the needle guard 132, such as to align the proximal hole 890 and the distal hole 892. A needle 108 can then pass through the distal hole 892 and retain the needle guard in the biased state or position without the tool. In the biased position, the second portion or hooking portion 805b of the hook contacts the catheter hub 102 and retains the catheter hub 102 from separation in combination with the catheter ramp 718. This makes it difficult for the intended user to remove the catheter hub 102 from the safety shield housing 700.

The needle 108 can be guided by a proximal hole 702h of the proximal side 702 of the safety shield housing 700, the proximal hole 890 of the needle guard 132, and optionally the top ramp 710. In the un-activated, biased position, the needle 108 can be further guided by the distal hole 892 and the catheter hub 102 and/or the catheter tube.

In embodiments, there may be additional ramps sized and shaped in the interior 720 of the safety shield housing 700 to hold or support the catheter hub 102 to prevent unwanted longitudinal or transverse movement of the catheter hub 102 from the safety shield housing 700. The additional ramps can also be incorporated to support the needle during retraction of the needle. The additional ramps can also be incorporated to ensure encapsulation of the needle within the safety shield housing by further limiting the needle's ability to pivot or rotate. The additional ramps can be molded within the cover or the housing structure or can be added, similar to adding one or more shims or tabs.

With reference to FIG. 11, a gap 805g is provided between the hook 805 and the interior surface of the top side 704. The gap 805g is provided as a clearance gap to enable the distal wall 804 to spring outwardly when no longer biased by the needle, such as after retraction of the needle tip proximally of the distal hole 892. The gap 805g should have a clearance or dimension larger than the thickness of the second portion 805b of the hook 805, or other structure of the hook if embodied in a different shape, to enable the second portion 805b to travel radially to clear the latching portion 102p. Once the hook 805 separates from the latching portion 102p, catheter hub can separate from the needle guard and the safety shield housing.

FIGS. 10 and 12 show the catheter assembly with the safety shield in an unbiased, activated position, but prior to separation of the catheter hub from the distal opening of the safety shield housing 700. When the needle hub 106 and the needle 108 are retracted from the proximal end of the catheter hub 102, such that the needle 108 is retracted from the distal hole 892, the needle guard 132 can be released from the biased position. The biased curve shaped portion 804b can return to an unbiased position, or to a less biased position, wherein the distance from the hook 805 to the intermediary wall 803 is longer than when in the biased position.

FIG. 12 shows the unbiased position of the needle guard 132 where the curve shaped portion 804b has returned to an unbiased position or is less biased than in the position of FIG. 11. When the sharp distal tip 110 of needle 108 is retracted, or moved proximally, into the needle guard 132 during the withdrawal of the needle, the needle 108 no longer extends through the distal hole 892 on the distal wall 804 of needle guard 132. The distal wall 804 of the needle guard 132 can flex to its unbiased, activated position.

During this unbiased movement of the needle guard 132, the hook 805 at the top of the distal wall 804 of needle guard 132 releases itself upwards from the catheter hub 102 to clear the latching portion 102p, allowing the catheter hub 102 to be disconnected from the needle guard 132.

As the distal hole 892 of the distal wall 804 of the needle guard 132 is no longer aligned with the longitudinal axis 891 of the needle 108, the distal wall 804 of the needle guard 132 can act as a barrier to the sharp distal tip 110 of the needle 108. The needle 108 can be contained within the needle guard 132 where the proximal hole of the safety shield catches the crimp 144 of the needle 108 while the distal wall 804 of the needle guard 132 acts as a barrier against distal movement of the needle tip 110 to re-expose the needle tip. In this protected position, the needle tip is trapped inside an enclosed space defined by the safety shield housing 700 and the distal wall 804.

The crimp 144 prevents the retracted needle 108 from retracting from the proximal hole 890, or the needle guard 132 and safety shield housing 700 from displacing distally off of the needle tip. As the needle 108 no longer constrains the safety shield in a biased position through the distal hole 892 in the position of FIG. 12, the longitudinal axis 893 of the distal hole 892 is offset from the longitudinal axis 891 of the proximal hole 802 and from the longitudinal axis of the needle 108. This prevents the needle from sliding forward past the distal wall 804 of the needle guard 132 after retracting from the distal wall 804.

With the needle guard 132 in the unbiased position, the catheter hub 102 is uncoupled, and is slidably movable relative to the needle guard 132 and the safety shield housing 700. This allows for separation of the catheter hub 102 from the safety shield housing 700. Accordingly, the catheter hub 102 can now be use for infusion or for drawing samples. In some examples, the catheter hub is provided with a valve and a valve opener, such as shown in FIGS. 1-3, so that fluid flow can be stopped by the valve until the slits of the valve are opened by the valve opener.

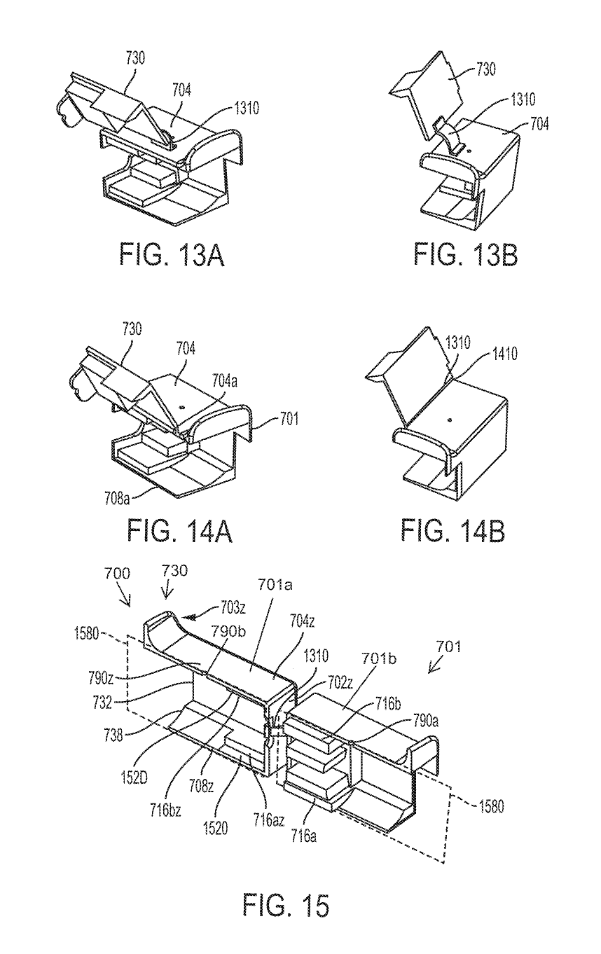

FIGS. 13A and 13B illustrate perspective views of an embodiment of a safety shield housing where the cover 730 and the housing structure 701 are coupled by a latch 1310.

FIG. 13A illustrates an embodiment where the cover 730 and the housing structure 701 are joined by a latch 1310. The latch 1310 can connect the cover side and the top side 704. The latch 1310 can be located at an intermediary location along the length of the top side 704. Alternatively, the latch 1310 can be near the distal end or the proximal end of the top side.

The latch 1310 can be defined by a thinner portion of material relative to the adjacent areas of the cover 730 and the housing structure 701. The thinner portion of material of the latch 1310 allows for a relatively weaker area of material that allows for flexure. As such, the latch 1310 can deform when force is applied to the cover 730 and the housing structure 701 to allow for rotation of one portion relative to the other.

In embodiments, the latch 1310 can be a material having different characteristics than at least one of the cover 730 and the housing structure 701. For example, the latch 1310 can be a polymer with a lower modulus of elasticity than the material of at least one of the cover 730 or the housing structure 701. In embodiments, the cover 730, the housing structure 701, and the latch 1310 can each be of different materials from one another. In still other examples, the latch can be an elastomer glued or welded to the cover and the top side. In an example, the latch 1310 can have the same material as the cover and the housing but is made from a thinner material or can include one or more notches or weakened sections or strips to facilitate bending.

According to methods of forming the safety shield housing 700, a single step injection molding can be performed where the different materials are co-molded or insert molded to form the safety shield housing 700 with different materials among the cover 730, the housing structure 701, and the latch 1310.

Embodiments can be formed by integrally molding of the cover 730, the housing structure 701, and the latch 1310. Alternatively, embodiments can have the cover 730 and the housing structure 701 formed separately and then joined by the latch 1310. The latch 1310 can be joined to the cover 730 and the housing structure 701 in such embodiments by means such as gluing, bonding, or welding.

Also, the latch 1310 can be a separate component. The cover 730 and the housing structure 701 can each have a throughhole configured to receive ends of the latch 1310. The ends of the latch can have retention components such that each end can be coupled to and retained in the throughholes of the cover 730 and the housing structure 701.

FIGS. 14A and 14B illustrate an embodiment of a safety shield housing where the cover 730 and the housing structure 701 are coupled by a latch 1310 that is a living hinge 1410. The cover 730, housing structure 701, and living hinge 1410 can be integrally formed by molding. The living hinge 1410 can be defined by a thinner portion of material relative to the adjacent areas of the cover 730 and housing structure 701. The thinner portion of material of the living hinge 1410 allows for a relatively weaker area of material that allows for flexure. As such, the living hinge 1410 can deform when force is applied to the cover 730 and the housing structure 701 to allow for rotation of one portion relative to the other

The living hinge 1410 can extend along an entire length of an edge 704a of the top side 704. Alternatively, the living hinge 1410 can extend only along a portion of the edge 704a. In embodiments where the living hinge 1410 extends only along a portion of the edge 704a, the living hinge 1410 can be substantially located at a proximal portion of the edge 704a, a distal portion of the edge 704a, or an intermediary portion of the edge 704a. The living hinge 1410 can extend less than half of the length of the edge 704a, half, or extend more than half of the length of the edge 704a.

In embodiments, the living hinge 1410 can couple the cover 730 and the housing structure 701 at an edge 708a of the bottom wall 708 instead. Alternatively, in embodiments where the cover 730 is a different wall of the safety shield housing 700, the living hinge 1410 can be attached to one of the edges of the adjacent walls of the housing structure 701 to which the cover contacts.

Alternatively, embodiments can have the cover 730 and the housing structure 701 substantially formed first separately and then joined by molding of the living hinge 1410 and surrounding areas of the cover 730 and housing structure 701.

In embodiments, the living hinge 1410 can be a material having different characteristics than at least one of the cover 730 and the housing structure 701. For example, the living hinge 1410 can be a polymer with a lower modulus of elasticity than the material of at least one of the cover 730 or the housing structure 701. In embodiments, the cover 730, the housing structure 701, and the living hinge 1410 can each be of different materials from one another.

FIG. 15 illustrates an embodiment of a safety shield housing 700 where the top wall, the bottom wall, and the proximal wall are split in two housing sections 701a, 701b. As shown, the safety shield housing is split along the lengthwise direction into two pieces. The two housing sections 701a, 701b are coupled by a latch 1310 coupling a proximal side 702z of one housing section 701a to a proximal side 702z of another housing section 701b. The safety shield housing 700 can be split along a central axial plane 1580 such that the two housing sections 701a, 701b are substantially equal in size. Alternatively, the safety shield housing 700 can be split along a line offset from the central axial plane 1580. In some examples, an analogous to the embodiments of FIGS. 13A, 13B, 14A, and 14B, one of the housing sections 701a, 701b, which may be called a first housing section 701a and a second housing section 701b, can be viewed as a cover 730 and the other housing section can be viewed as a housing structure 701.

Each half of the housing structure can include a top side 704z, a proximal side 702z, a distal side 703z, and a bottom side 708z. On interior surfaces of the top side 704z and the bottom side 708z, there can be provided stop pieces 716az, 716bz. The stop pieces 716az, 716bz can be inset from edges of the top side 704z and the bottom side 708z by a mating region 1520. The stop pieces 716a, 716b, the top ramp 710, and the bottom ramp 712 of the second housing section 701b can protrude beyond the central axial plane 1580 of the housing structure 701b. As such, the protruding portions of the stop pieces 716a, 716b, the top ramp 710, and the bottom ramp 712 can fit into the mating region 1520 when the covering 730 and the housing structure 701 are fitted together in an assembled state.

The hole 790 can be split into arcuate portions 790a of the second housing structure 701b and 790b of the first housing structure 701a. In embodiments, the hole 790 can be offset from the divide between the two housing sections 701a, 701b so that the hole 790 is located completely on one of the housing sections 701a, 701b.

The latch 1310 can be composed on a material suitable for the movement of bringing the two housing sections 701, 701b into an assembled position. The latch 1310 can have an arcuate portion similar to the latch 1310 of FIGS. 13A and 13B. The latch 1310 can be comparatively thinner than the thickness of the sides of the two housing sections 701a, 701b. In embodiments, the latch 1310 can be attached to the two housing sections 701a, 701b at a position near the central axial plane 1580.

In embodiments, the latch 1310 can be a material having different characteristics than at least one of the two housing sections 701a, 701b. For example, the latch 1310 can be a polymer with a lower modulus of elasticity than the material of at least one of the two housing sections. In embodiments, the two housing sections and the latch 1310 can each be of different materials from one another.

Embodiments can be formed by integral molding of the latch 1310 with the two housing sections 701a, 701b. The method can include conventional molding techniques, can include co-molding, can include insert molding.

Alternatively, embodiments can have the housing sections 701a, 701b formed separately and then joined by the latch 1310. The latch 1310 can be joined to the two housing sections 701a, 701b in such embodiments by means such as molding or plastic welding. Also, the latch 1310 can be a separate component. The two housing sections 701a, 701b can each have a throughhole in the respective proximal sides 702, 702z configured to receive ends of the latch 1310. The ends of the latch can have retention components such that each end can be coupled to and retained in the throughholes of the two housing sections 701a, 701b.

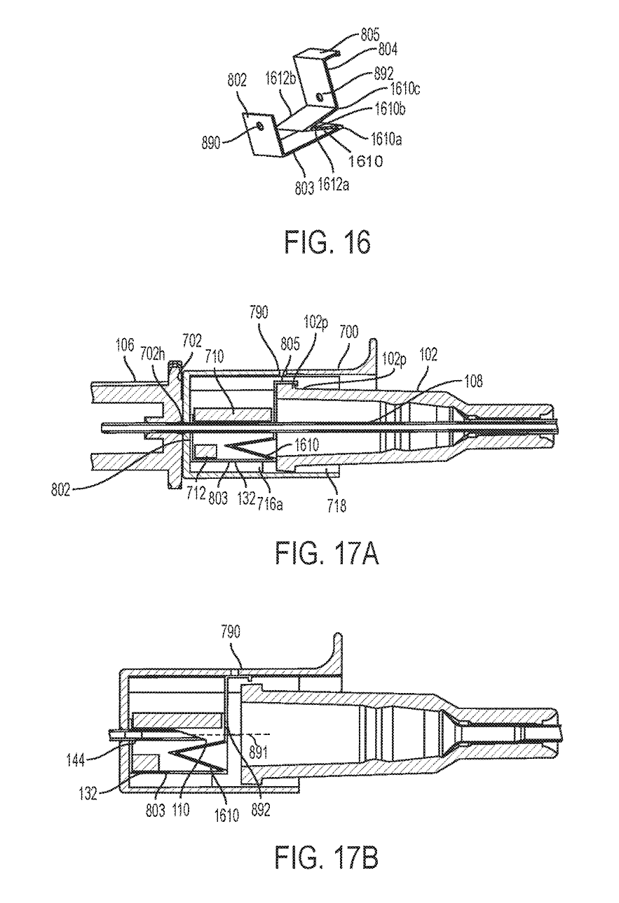

The various alternative safety shield housings described with reference to FIGS. 13A-15 can be used to house a needle guard described elsewhere herein, such as the needle guard of FIG. 8 or 16. Features in the alternative safety shield housings for accommodating a needle guard, such as incorporating slots or gaps, can be similar to those described elsewhere herein.

FIG. 16 illustrates an embodiment of the needle guard 132 with a folded biasing portion 1610 connected to the distal wall 804. The needle guard 132 of the present embodiment is similar to the needle guard of FIG. 8 with a few exceptions. In the present embodiment, the folded biasing portion 1610 can be formed from a plurality of folds between the distal hole 892 and the intermediary wall 803.

The proximal wall 802 can have a proximal hole 890 and the distal wall 803 can have a distal hole 892. The plurality of folds create a spring portion. The folds can have sharp angled folds or can be undulating like a sine wave. The folded biasing portion 1610 can include a first acute angle 1610a between the intermediary wall 803 and a first fold portion 1612a, a second acute angle 1610b, a second fold portion 1612b, and an obtuse angle 1610c between the distal wall 804 and the second fold portion 1612b. In other examples, the folded biasing portion 1610 can include additional fold portions and additional acute angles.

The first acute angle 1610a and the second acute angle 1610b can be biased into smaller angle biased positions and can allow for compression of the distal wall to shorten the distance between the distal hole 892 and the intermediary wall 803.

The distal wall 804 can have a shape and size such that it is able to flex between a biased position and an unbiased position. The distal wall 804 can have the ability to flex such that the end of the distal wall 804 joined with the hook 805 can be deflected with movement along a plane substantially perpendicular to the longitudinal axis of the needle.

Similarly to FIG. 8, the proximal hole 890 and the distal hole 892 can be arranged such that in a relaxed, unbiased state of the needle guard 132, a longitudinal axis 891 of the proximal hole 890 is offset from a longitudinal axis 893 of the distal hole 892. The proximal hole 890 and the distal hole 892 can be arranged such that when the needle guard 132 is in a biased, or compressed state, the proximal hole 890 and the distal hole 892 can be aligned such that the longitudinal axis 891 of the proximal hole 890 aligns with the longitudinal axis 893 of the distal hole 892.

Although the needle guard 132 of the present embodiment is shown as being assembled with a safety shield housing 700 (FIG. 17A), it is also understood that the needle guard 132 of FIG. 16 can be assembled to a catheter assembly 100 without a safety shield housing 700. The needle guard 132 can be applied in a suitably sized and shaped catheter hub 102, similar to that of FIG. 1, or needle hub 106 without the safety shield housing 700. For example, such a catheter hub 102 can have an interior catch for coupling with the needle guard 132.

FIGS. 17A and 17B illustrate cross sectional views of an assembled catheter assembly with the safety shield housing 700 and needle guard 132 illustrated by FIG. 16. The assembled catheter assembly can have substantially similar considerations as the catheter assembly described in FIGS. 11 and 12 and elsewhere. The bottom ramp 712 can be sized to provide clearance for the folded biasing portion 1610 of the needle guard 132. As such, the bottom ramp 712 can be smaller than the top ramp 710.

FIG. 17A shows the needle guard 132 retained in a biased position by the needle 108 extending through the proximal hole 890 and the distal hole 892. In the un-activated position, the hook 805 couples to the latching portion 102p of the catheter hub 102, thereby preventing separation of the catheter hub 102 from the safety shield housing 700.

The ramps 710, 712 of the housing can act as guides for the needle 108 and also provide a slot for the placement of the needle guard 132. The intermediary wall 803 of the needle guard 132 can be positioned between the bottom ramp 712 and one of the stop pieces 716a. The proximal wall 802 of the needle guard 132 can be positioned between the top and bottom ramps 710, 712 and the proximal side 702 of the safety shield housing 700. The proximal wall 802 and the intermediary wall 803 of the needle guard 132 can be firmly fitted between the ramps 710, 712, proximal side 702, and the stop piece 716a.

The top ramp of the housing can also prevent the sharp distal tip 110 of the needle 108 shown in FIG. 17B being able to be tilted upwards towards the distal hole on the distal wall of the safety shield in an unbiased position to re-expose the needle following needle protection. Alternatively, the ramps 710, 712 can be formed and molded on the cover 730 instead of the housing structure 701 side of the safety shield housing 700. The two ramps can be have different shapes to accommodate the folded biasing portion 1610 of the needle guard. As shown, the lower ramp 712 does not distally as far as the upper ramp 710.

The ramps 710, 712 can be integrally molded as a single component together with the housing structure 701 of the safety shield housing 700. The ramps 710, 712 can be molded in a separate step from the molding of either the housing structure 701 or the cover 730.

The proximal wall 802 and the intermediary wall 803 can be substantially perpendicular to one another or can be other than right angle to create a bias against the safety shield housing when the needle guard is installed inside safety shield housing. In this way, the needle guard 132 can be retained in the safety shield housing 700 in the rear gap between the ramps and the housing proximal wall. A portion of the length of the intermediary wall 803 can be retained by the bottom ramp 712 and one of the stop pieces 716a, such as the stop piece on the bottom wall. Alternatively, the proximal wall 802 and the intermediary wall 803 can meet with an oblique angle. The bottom ramp 712 can be correspondingly sized and shaped to maintain retention of the needle guard 132.

The distal wall 804 of the needle guard 132 can be positioned in the distal region 724 of the safety shield housing 700, such that the distal wall 804 can be biased and unbiased.

In the un-activated, biased position, the folded biasing portion 1610 of the needle guard 132 is biased by the needle 108. In this way, the folded biasing portion 1610 is flexed, or biased, such that the distance from the hook 805 to the intermediary wall 803 is shorter than when the needle guard 132 is unbiased, or not constrained by the needle. As shown, the access hole 790 can be positioned above the hook 805. A tool can be inserted into the hole 790 of the top side 704 to bias the needle guard 132 such that the needle 108 can pass through the distal hole 892 in addition to the proximal hole to bias the needle guard. In the biased position, the hooking portion 805b contacts the latching portion 102p of the catheter hub 102 and retains the catheter hub 102 from separation from the needle guard and the needle shield housing. This makes it difficult for the user to remove the catheter hub 102 from the safety shield housing 700 prior to activating or releasing the needle guard from the biased state.

The needle 108 can be guided by a proximal hole 702h of the proximal side 702 of the housing 700, the proximal hole 890 of the needle guard 132, and the top ramp 710. In the un-activated, biased position, the needle 108 can be further guided by the distal hole 892 and the catheter hub 102.

In embodiments, there may be additional ramps sized and shaped in the interior 720 of the safety shield housing 700 to hold or support the catheter hub 102 to prevent unwanted longitudinal or transverse movement of the catheter hub 102 from the safety shield housing 700. The additional ramps may be similar to shims used for adjusting the various components to take up slacks or gaps.

FIG. 17B shows the catheter assembly with the needle guard in the unbiased, activated position, prior to separating the catheter hub from the safety shield housing. When the needle hub 106 and the needle 108 are retracted from the proximal end of the catheter hub 102, such that the needle 108 is retracted from the distal hole 892, the needle guard 132 can be released from the biased position. The folded biasing portion 1610 can return to an unbiased position, or less biased position, wherein the distance from the hook 805 to the intermediary wall 803 is longer than the biased position.

During this unbiased movement, the hook 805 at the top of the distal wall 804 of needle guard 132 moves upwardly of the latching portion of the catheter hub 102, allowing the catheter hub 102 to be disconnected from the needle guard 132.

As the distal hole 892 of the distal wall 804 of the needle guard 132 is no longer aligned with the longitudinal axis 891 of the needle 108, the distal wall 804 of the needle guard 132 can act as a barrier to the sharp distal tip 110 of the needle 108. The needle 108 can be contained within the safety shield where the proximal hole of the safety shield catches the crimp 144 of the needle 108 while the distal wall 804 of the needle guard 132 provides a barrier for the tip 110 of the needle 108.

The crimp 144 prevents the retracted needle 108 from moving proximally of the proximal hole 890 or the needle guard to displace distally off of the needle tip. As the needle 108 is no longer retaining the safety shield in a biased position through the distal hole 892, the longitudinal axis 893 of the distal hole 892 is offset from the longitudinal axis 891 of the proximal hole and the longitudinal axis of the needle 108. This prevents the needle from sliding forward past the distal wall 804 of the needle guard 132 after retracting from the distal wall 804.

With the needle guard 132 in the unbiased position, the catheter hub 102 is uncoupled, and can slidably move relative to the needle guard 132 and the safety shield housing 700. This allows for separation of the catheter hub 102 from the safety shield housing 700. Accordingly, the catheter hub 102 can now be ready for infusion. The catheter hub can include a valve and a valve opener as shown in FIGS. 1-3.

FIG. 18 illustrates an embodiment of a needle guard 132 having a design derived from the safety shield spring clip disclosed in E.P. Patent No. 1,702,643, which is expressly incorporated herein by reference in its entirety.

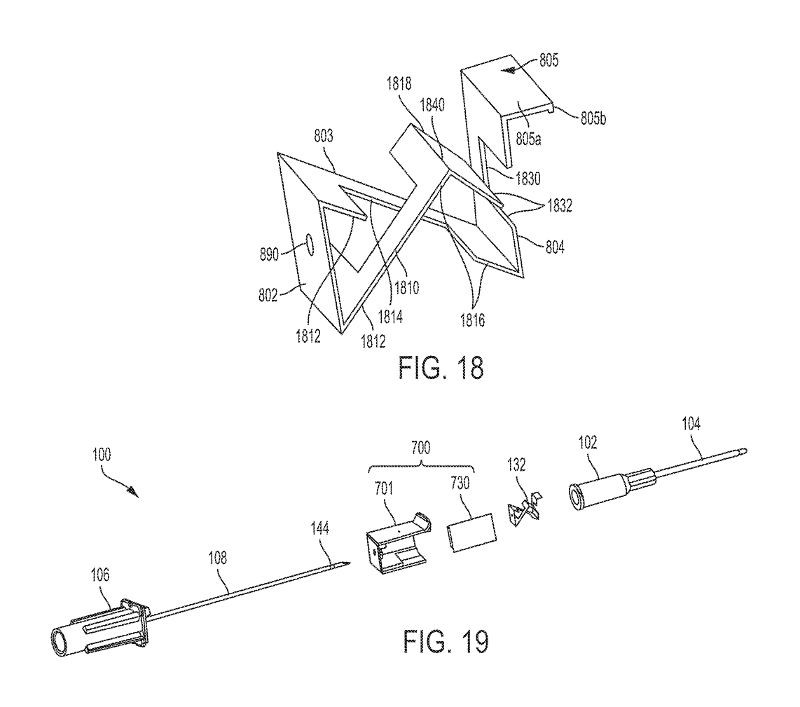

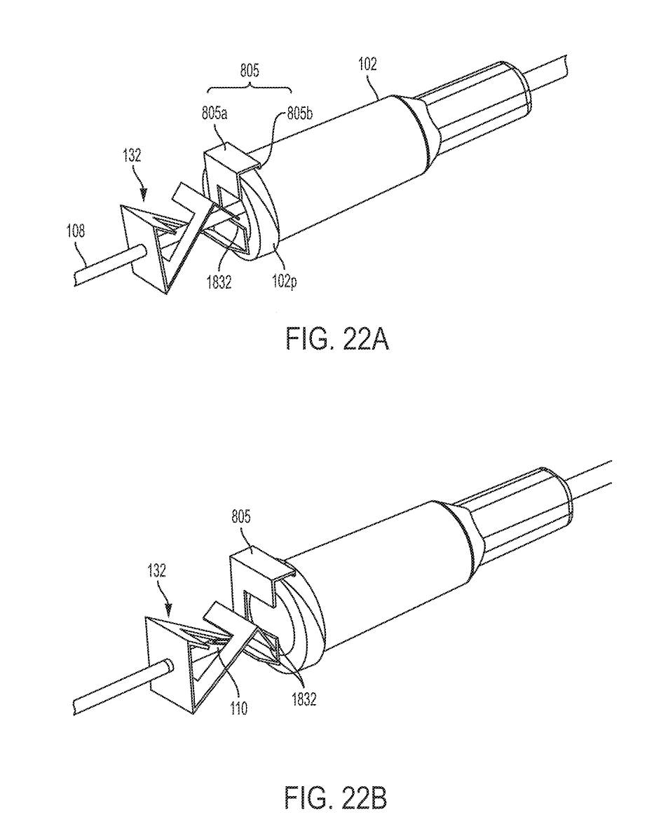

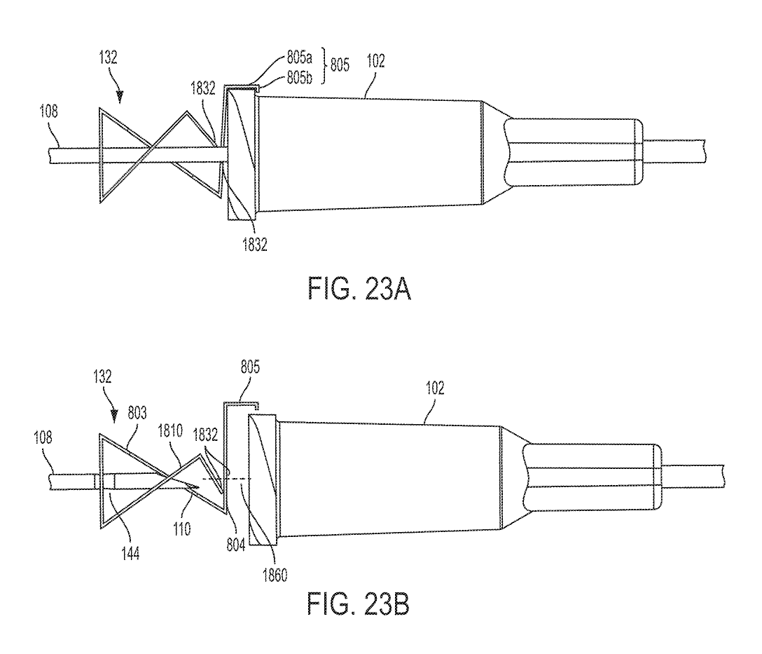

The needle guard 132 of the present embodiment can have two resilient arms defined by intermediary walls 803, 1810. The two intermediary walls 803, 1810 of the two arms can be respectively joined at their proximal ends in a hinged arrangement to the ends of a proximal wall 802. With reference to FIGS. 19-23B, the proximal wall 802 can be configured to be substantially perpendicular to a longitudinal axis of the needle 108. The proximal wall 803 can have a proximal hole 890 having a diameter that is slightly larger than an outer diameter of the needle 108 configured to pass through the proximal hole 890 but smaller than a crimp or change in profile incorporated with the needle.

The intermediary walls 803, 1810 of the two arms can each include a proximal wide portion 1812, an intermediary narrow portion 1814, and a distal wide portion 1816. The two arms can act as cantilever springs. The intermediary walls 803, 1810 or the two arms can be intermediary narrow portions 1814 on opposed lateral sides, such that the intermediary walls 803, 1810 of the two arms can cross one another.

One of the intermediary walls 803 can attach to the distal wall 804. The distal wall 804 can have a cut out portion 1830 with a lateral clamping edge 1832. The distal wall 804 can be attached to a hook 805. The hook 805 can be composed of a first portion 805a and a hooking portion 805b. The hook 805 can be configured to allow for hooking or latching on to a Luer thread on a catheter hub or a corresponding latching portion 102p formed on the catheter hub. Such a detent formed on the catheter hub can take the form of a flange, Luer locking ears, a notch, a slot or a peg.

The first portion 805a can be configured to extend in a direction towards a distal end of the catheter hub in order for the hooking portion 805b to clear the corresponding Luer thread or detent of the catheter hub.

The hook 805 can have different geometries as desired and necessary for interfacing with a corresponding catheter hub. In embodiments, the hook 805 can be triangular in shape with an upward angled first portion 805a and a downward angled hooking portion 805b. In embodiments, the hook 805 can have a hooking portion 805b without a first portion 805a. For example, the hook 805 can be comprised of an arcuate portion or a curved portion.

The other intermediary wall 1810 can have an angled distal end section 1818 The distal end of the angled distal end section 1818 can have a lateral clamping edge 1832. The angled distal end section 1818 can be angled in a direction towards the cutout portion 1830.

The safety shield can be made of a stamped metal sheet that is then folded into the desired designed shape wherein, in the biased position, the safety shield engages the catheter hub and in the unbiased position the safety shield disengages the catheter hub.

Although the needle guard 132 is shown as being assembled with the safety shield housing 700, it is also understood that the needle guard 132 of FIG. 18 can be used with a catheter assembly 100 without a safety shield housing 700. The needle guard 132 can be applied in a suitably sized and shaped catheter hub 102, similar to that of FIG. 1, or needle hub 106 without the safety shield housing 700. For example, such a catheter hub 102 can have an interior catch for coupling with the needle guard 132.

FIG. 19 illustrates an exploded view of an embodiment of a catheter assembly 100 of the present disclosure. Additional components not illustrated in FIG. 19 but understood from the incorporated references or from earlier disclosed embodiments can be added to the catheter assembly 100 as desired.

The catheter assembly 100 generally comprises a catheter or catheter tube 104 and a catheter hub 102 at a distal end of the catheter assembly 100. A distal end of the safety shield housing 700 with a housing structure 701 and a cover 730 can contact a proximal end of the catheter hub 102. The safety shield housing 700 can house the needle guard 132. This is in contrast with the embodiment illustrated in FIG. 1 wherein the needle guard 132 is housed in the catheter hub 102. In this way, the catheter hub does not have the design constraints of having to be sized and shaped to receive and house a safety shield. The needle hub or cannula hub 106 can attach to a proximal end of the needle 108 and can contact a proximal end of the needle guard 132 when assembled thereto in the ready to use position.

As described above, embodiments of the components from the incorporated references can be implemented for the various components disclosed herein. For example, regarding the catheter hub, it is possible to practice a wingless catheter hub, a winged catheter hub, a ported catheter hub, or a catheter adapter with an inner lumen with a valve or permanent septum.

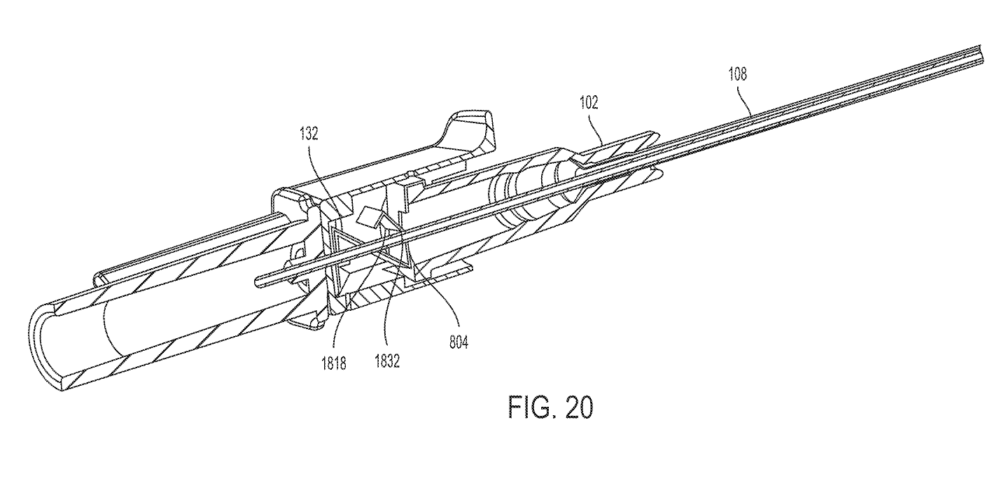

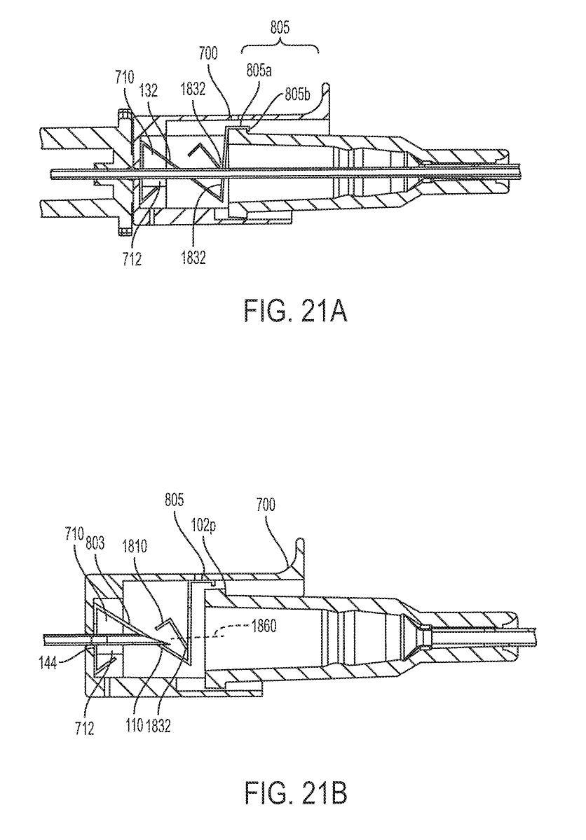

FIGS. 20, 21A, 21B, 22A, 22B, 23A, and 23B illustrate various views of the safety shield in an un-activated, biased position and in an activated, unbiased position as would be seen in an assembled catheter assembly. FIGS. 20, 21A, 22A, and 23A illustrate the safety shield in the un-activated, biased position. FIGS. 21B, 22B, and 23B illustrate the safety shield in the activated, unbiased position. The assembled catheter assembly can have substantially similar considerations as the catheter assembly described in FIGS. 11, 12, and 17A-17B.

As shown in FIGS. 20, 21A, 22A, and 23A, the needle 108 can bias the intermediary walls 803, 1810 such that the needle 108 can extend into the catheter hub 102. In this case, lateral clamping edges 1832 on each of the distal wall 804 and the angled distal end section 1818 can rest on the needle 108 in the biased position.

The needle guard 132 can be held in place by fitment or containment in the safety shield housing 700 with the top ramp 710, bottom ramp 712, and interior walls of the safety shield housing 700 retaining the proximal wall of the needle guard, as previously discussed. The top ramp 710 can be sized and shaped, such as a trapezoid, to fit with the needle guard 132. The bottom ramp can be sized and shaped, such as a rectangle, to fit with the needle guard 132.

In the un-activated, biased position, the needle 108 can retain and bias the intermediary wall resilient arms 803, 1810. The shaft of the needle 108 passes through the proximal hole 890 and the lateral clamping edges 1832 on each of the distal wall 804 and the angled distal end section 1818 can rest on the needle in the biased position. In this position, where the distal wall 804 is biased relatively downward, the hook 805, with a first portion 805a and a hooking portion 805b, can latch onto a latching portion 102p of the catheter hub 102.

As shown in FIGS. 21B, 22B, and 23B, in the activated, unbiased position, the angled distal end section 1818 can contact the distal wall 804 and block a direct sight path from the proximal hole 890 to the cut out portion 1830.

When the sharp distal tip 110 of needle 108 is retracted, or moved, into the needle guard 132 during the withdrawal of the needle, the needle 108 no longer extends through the cut out portion 1830 on the distal wall 804 of needle guard 132. The distal wall 804 of needle guard 132 can flex to its unbiased, activated position.

During this unbiased movement, the hook 805 at the top of the distal wall 804 of needle guard 132 releases itself upwards from the catheter hub 102, allowing the catheter hub 102 to be disconnected from the needle guard 132.

As the cut out portion 1830 of the distal wall 804 of the needle guard 132 has a lowest opening 1860 that prevents clearance of the needle 108, the distal wall 804 of the needle guard 132 can act as a barrier to the sharp distal tip 110 of the needle 108. The needle 108 can be contained within the safety shield where the proximal hole of the safety shield catches the crimp 144 of the needle 108 while the distal wall 804 of the needle guard 132 catches the tip 110 of the needle 108. The crimp 144 prevents the retracted needle 108 from retracting from the proximal hole 890. Additionally, the distal end section 1818 can further prevent the needle from moving forward as it blocks a direct sight path from the proximal hole 890 to the distal wall 804.

With the needle guard 132 in the unbiased position, the catheter hub 102 is uncoupled, and is slidably movable relative to the needle guard 132 and the safety shield housing 700. This allows for separation of the catheter hub 102 from the safety shield housing 700. Accordingly, the catheter hub 102 can be in a ready to use configuration.

Although the needle guard 132 is shown as being assembled with the safety shield housing 700, it is also understood that the needle guard 132 of FIG. 19 can be used with a catheter assembly 100 without a safety shield housing 700. The needle guard 132 can be applied in a suitably sized and shaped catheter hub 102, similar to that of FIG. 1, or needle hub 106 without the safety shield housing 700. For example, such a catheter hub 102 can have an interior catch for coupling with the needle guard 132.

The present disclosure provides for a catheter assembly having a catheter hub having a catheter; a safety shield or needle guard; a safety shield housing; and a needle hub having a needle; wherein the safety shield housing is disposed between the catheter hub and the needle hub; wherein the safety shield is configured to couple with a proximal portion of the catheter hub. The safety shield can be biased by the needle to then couple to the proximal portion of the catheter hub.

The present disclosure provides for a catheter assembly having a catheter hub having a catheter; a safety shield or needle guard; a safety shield housing; and a needle hub having a needle; wherein the safety shield housing is disposed between the catheter hub and the needle hub; wherein the safety shield comprises a hook housed inside the safety shield housing; wherein the hook latches onto a proximal portion of the catheter hub.

Methods of making catheter assemblies and of using the catheter assemblies as described herein are within the scope of the present invention. The methods are understood to include the use of needle guards and safety shield housings with an integrated IVC, which has a side fluid port extending from a catheter hub with a tubing and a fluid adapter at the end of the tubing.

Although limited embodiments of safety shields, safety shield housings and their applications in catheter assemblies have been specifically described and illustrated herein, many modifications and variations will be apparent to those skilled in the art. For example, the various features of a safety shield housing may incorporate alternate materials, etc. Furthermore, it is understood and contemplated that features specifically discussed for one safety shield and safety shield housing embodiment may be adopted for inclusion with another safety shield embodiment and/or safety shield housing, provided the functions are compatible. Accordingly, it is to be understood that the safety shields, safety shield housings, and their applications in catheter assemblies constructed according to principles of the disclosed devices, systems, and methods may be embodied other than as specifically described herein. The disclosure is also defined in the following claims.

What is claimed is:

1. A catheter assembly comprising:
a catheter hub comprising a catheter;
a needle guard;
a safety shield housing having an interior having the needle guard located therein; and
a needle hub comprising a needle with a needle tip;
wherein the safety shield housing is disposed between and couples the catheter hub and the needle hub and the needle guard engaging the catheter hub.

2. The catheter assembly according to claim 1, wherein the safety shield housing receives a portion of the catheter hub at a distal opening of the safety shield housing.

3. The catheter assembly according to claim 2, wherein the needle guard comprises a hook and the hook engages a proximal portion of the catheter hub.

4. The catheter assembly according to claim 3, wherein the hook has at least one of a curved surface or a lip for engaging the catheter hub.

5. The catheter assembly according to claim 1, wherein the needle guard comprises a resilient spring.

6. The catheter assembly according to claim 1, wherein the needle guard comprises a proximal hole and a distal hole spaced from one another by an intermediary wall, the proximal hole and the distal hole being sized to accept the needle.

7. The catheter assembly according to claim 6,
wherein the needle guard comprises a resilient spring that is biased in a position where the needle is inserted through the proximal hole and the distal hole; and
wherein the resilient spring of the needle guard is unbiased in a position where the needle is displaced out of the distal hole.

8. The catheter assembly according to claim 7, wherein a longitudinal axis of the distal hole is offset from a longitudinal axis of a proximal hole when the needle guard is unbiased.

9. The catheter assembly according to claim 1, wherein the needle guard has a first part and a second part and wherein the first part of the needle guard is located in a slot between a ramp and a bottom side of the safety shield housing.

10. The catheter assembly according to claim 9, wherein the second part of the needle guard is located in a slot between the ramp and a proximal side of the safety shield housing.

11. The catheter assembly according to claim 1, wherein the needle guard comprises a proximal hole, a distal hole, a hook, and a folded biasing portion comprising at least two acute angles or arcuate surfaces.

12. The catheter assembly according to claim 1, wherein the catheter hub comprises a side fluid port and a tubing connected to the side fluid port.

13. The catheter assembly according to claim 12, further comprising a valve or a septum located inside the catheter hub.

14. The catheter assembly according to claim 13, further comprising a fluid adapter connected to the tubing.

15. A method of assembling a catheter assembly, the method comprising:
coupling a safety shield housing with a catheter hub comprising a catheter tube;
inserting a needle guard comprising a resilient spring into an interior of the safety shield housing; and
coupling a needle hub comprising a needle with the safety shield housing;
wherein the safety shield housing is disposed between and couples the catheter hub and the needle hub and the needle guard engaging the catheter hub.

16. The method according to claim 15, wherein the safety shield housing receives a portion of the catheter hub through a distal opening of the safety shield housing.

17. The method according to claim 16, wherein the needle guard comprises a hook engaging a proximal portion of the catheter hub.

18. The method according to claim 17, wherein the hook has at least one of a curved surface or a lip for engaging the catheter hub.

19. The method according to claim 15, wherein the needle guard comprises a proximal hole and a distal hole, the proximal hole and the distal hole being sized to accept the needle.

20. The method according to claim 19,
wherein the needle guard comprises a resilient spring that is biased in a position where the needle is inserted through the proximal hole and the distal hole; and
wherein the resilient spring of the needle guard is unbiased in a position where the needle is displaced out of the distal hole.

21. The method according to claim 20, wherein a longitudinal axis of the distal hole is offset from a longitudinal axis of a proximal hole when the needle guard is unbiased.

22. The method according to claim 21, wherein the needle guard comprises a resilient spring.

23. The method according to claim 22, wherein the resilient spring is a curve shaped portion of a distal wall, is a folded biasing portion comprising an acute angle or a curved surface, or a cantilever spring.

24. The method according to claim 15, wherein the catheter hub comprises a side fluid port and a tubing connected to the side fluid port.
